(19) United States
(12) Patent Application Publication
(10) Pub. No.: US 2010/0048462 A1
(43) Pub. Date: Feb. 25, 2010

(54) TRUNCATED PTH PEPTIDES WITH A CYCLIC CONFORMATION

(75) Inventors: Trine Skovlund Ryge, Frederikssund (DK); Martin Stahlhut, Rodovre (DK); Carsten Boye Knudsen, Greve (DK); Bjarne Due Larsen, Roskilde (DK)

Correspondence Address: CLARK & ELBING LLP, 101 FEDERAL STREET, BOSTON, MA, 02110, US

(73) Assignee: Zealand Pharma A/S, Glostrup (DK)

(21) Appl. No.: 12/518,171

(22) PCT Filed: Dec. 6, 2007
(86) PCT No.: PCT/GB2007/004664

§ 371 Date: Oct. 16, 2009

Related U.S. Application Data
(60) Provisional application No. 60/873,723, filed on Dec. 8, 2006.

(30) Foreign Application Priority Data
Dec. 8, 2006 (EP) 06025423.2

(51) Int. Cl. A61K 38/12; A61K 38/10; C07K 7/64; C07K 7/08

(52) U.S. Cl. 514/9; 514/13; 530/317; 530/326

(57) ABSTRACT

The present invention provides PTH peptides which are cyclised substitution analogues of the truncated PTH fragment PTH (1-17) and which preferably retain the desired or similar biological activity of human PTH (1-34).

### FIELD OF THE INVENTION

The present invention relates to substitution analogues of peptide PTH(1-17) with a cyclic structural feature, to methods of preparing the analogues and their medical use.

### BACKGROUND OF THE INVENTION

### Parathyroid Hormone

Parathyroid hormone (PTH), an 84 amino acid peptide, is the principal regulator of ionized blood calcium in the human body (Kronenberg, H. M., et al., In Handbook of Experimental Pharmacology, Mundy, G. R., and Martin, T. J., (eds), pp. 185-201, Springer-Verlag, Heidelberg, 1993). It is also known that full length PTH is anabolic to bone when administered intermittently (Dempster, D. W., et al., Endocr. Rev., 14: 690-709, 1993).

PTH(1-34) and PTH(1-84) have been shown to efficiently increase bone mineral density and bone strength in animal studies. Furthermore, treatment of osteoporotic patients with these PTH variants reduces the incidence of new osteoporotic fractures (Greenspan, S. L. et al., Ann. Intern. Med., 146: 326-339, 2007 and Neer, R. M. et al. N. Engl. J. Med. 344: 1434-1441, 2001).

Although treatment with PTH(1-84) and PTH(1-34) stimulates bone strength and prevents fractures, tolerability is limited by transient mobilization of calcium and hypercalcemia following each dosing which is commonly associated with nausea. Furthermore, these peptides are not orally or transmucally available, but have to be injected daily.

In addition, shortened PTH analogues have repeatedly failed to evoke anabolic effects on bone (Murrills, R. J. et al., Bone 35: 1263-1272, 2004 and Rhee, Y. et al., Yonsei Med. J., 47: 214-222, 2006). An exception is the cyclic, but still relatively large C-terminally truncated analogue ostabolin (hPTH(1-31)) (Whitfield, J. F. et al., Calcif. Tissue Int., 60: 26-29, 1997).

### Osteoporosis

Postmenopausal osteoporosis is a skeletal disorder characterized by a reduction in bone density and strength, associated with an increased risk of fracture (Lane et al., Clin. Orthop. Relat, Res., 139-50, 2000; Christiansen, Bone, 17: 513S-6S, 1995). Osteoporotic fractures most often occur in the vertebrae, the hips or the femoral neck. These fractures severely impair the patients' quality of life because of pain, long-lasting immobility and poor recovery.

Bone is a highly active tissue in the human body. Bone is continuously remodelled by two types of cells: bone resorbing osteoclasts and bone forming osteoblasts. When bone resorption exceeds bone formation, bone loss occurs that may develop into osteoporosis (Seeman and Delmas, N. Engl. J. Med. 354: 2250-61, 2006). Osteoporosis is frequently first diagnosed when a fracture has occurred.

Postmenopausal estrogen deficiency is the most common cause of the disease, as estrogen puts a break on osteoclast lifespan. Other major risk factors in the development of osteoporosis include: low calcium intake, vitamin D deficiency, type 1 diabetes, rheumatoid arthritis, long-term use of medication such as anticonvulsants and corticosteroids and low levels of testosterone in men.

### Signaling and Bone-Anabolic Activity of PTH Analogues

PTH acts on the PTH/PTHrP receptor (PTH1R), a class II G protein-coupled seven trans-membrane domain receptor that couples to adenylyl cyclase/cAMP (Jüppner, H. et al., Science, 254:1024-1026, 1991). Other signalling pathways of this receptor, such as elevation of intracellular calcium, phospholipase C-dependent and -independent activation of protein kinase C, have been described. Deletion analysis studies have shown that the amino-terminal residues of PTH play a crucial role in stimulating the PTH1R to activate the cAMP and IP3 signalling pathways. Signalling through the PTH1R seems to be dependent on a variety of parameters, including cell type, receptor density and others. The signalling mechanisms leading to biological activity on bone have not been fully elucidated yet. It is believed that PTH1R cAMP-signalling through cAMP is necessary, but not sufficient, for the anabolic effects of PTH analogues on bone.

Accordingly, full-length PTH (PTH(1-84)) and the well-known, fully-active fragment PTH(1-34) have, administered intermittently, clinically confirmed anabolic activity on bone (Grenspan, S. L. et al., Ann. Intern. Med. 146:326-339, 2007; Neer, R. M., et al., N.E.J.M., 344: 1434-1441, 2001). By contrast, the search for smaller analogues with bone-anabolic properties has been largely unsuccessful. C-terminally truncated analogues with a length of at least 28 amino acids have been shown to be anabolic in animal models of osteoporosis (Whitfield J. F. et al., J. Bone Miner. Res., 15: 964-970, 2000). However, further truncation has led to the complete loss of the bone-anabolic activity, even when agonist activity on the cAMP pathway of the PTH1R was retained (Murrills R. J. et al., Bone, 35: 1263-72, 2004).

Although short analogues consisting of as little as 11 amino acids can activate the PTH1R with low potency (WO 04/067021), bone-anabolic activity of these analogues has not been reported and would not be expected.

In WO 03/009804 and WO 04/093902, it is proposed that introduction of α-helix stabilizing amino acids in position 1 and 3 of a PTH(1-14) analogue improves the ability of the compounds to stimulate cAMP accumulation. The most potent compound identified was [Ac₅c¹, Aib³, Gln¹⁰, Har¹¹, Ala¹², Trp¹⁴]PTH(1-14) ([Acₛc¹, Aib³]MPTH(1-14)). The bone-anabolic activity of these compounds is, however, not shown. However, a closely related analogue [Aib^{1.3}, Phe⁷, Nle⁸, Arg¹¹, Ala¹², Trp¹⁴]PTH(1-14) did not exhibit any bone-anabolic activity on the bones of ovariectomized rats although the peptide activated the PTH1R in vitro (Rhee, Y. et al., Yonsei Medical Journal, 47: 214-222, 2006). Moreover, the bone-anabolic activity of PTH(1-29) has been shown to be approximately 20-fold less potent than PTH(1-34) in the ovariectomized rat model, while a modified form of PTH(1-21) ([Ala^{1.3}, Nle⁸, Gln¹⁰, Har¹¹, Trp¹⁴, Arg¹⁹, Tyr²¹]rPTH(1-21) (MPTH(1-21)) was inactive (Murrills, R. J. et al., (2004) Bone, 35, 1263-1272). In conclusion, agonist activity on cAMP-signalling pathway of the PTH1R in vitro alone is not at all predictive for bone-anabolic activity in vivo.

### The Cytochrome P450 Enzyme System

The cytochrome P450 (CYP) enzyme system consists of more than 50 human isoforms of which five (CYP1A2, CYP2C9, CYP2C19, CYP2D6 and CYP3A4) are responsible for the metabolism of 95% of drugs metabolized by the CYP system (P. Anzenbacher and E. Anzenbacherova, Cell. Mol. Life. Sci., 58: 737-47, 2001). The co-administration of drugs that are metabolized by and/or inhibit the CYP system, can lead to accumulation of drugs and/or intermediate toxic metabolites in the body and thereby inducing serious side effects. Accordingly, the FDA recommends characterization of CYP interactions of all new chemical entities (guidance for industry “drug metabolism drug interaction studies in the drug development process: studies in vitro” U.S. Food and Drug Administration, April 1997). One of the CYP isoforms, CYP2D6, is anticipated to be responsible for the metabolism of 25% of all CYP metabolized drugs. The serious potential of CYP2D6 inhibition is the observed cardiotoxicity of thioridanzine, which demonstrates the potential risk of drugs associated with CYP2D6 inhibition (Llerena A. et al., J. Phychopharmacol., 16(4): 361-4, 2002).

### SUMMARY OF THE INVENTION

Broadly, the present invention provides PTH peptides which are cyclised substitution analogues of a C-terminally truncated PTH fragment, for example PTH(1-17), and which preferably retain a desired biological activity of human PTH (1-34). In some embodiments of the invention, the cyclic PTH peptides are provided in the form of dimers. Alternatively or additionally, the present invention provides PTH analogues with low interference with CYP450 enzyme and/or bone-anabolic activity that leads to the formation of mineralized bone in adult vertebrates. The relatively small size of the peptide analogues of the present invention as compared with PTH(1-34) may be utilized in formulations for oral, nasal or pulmonary administration.

Throughout this specification, residue positions are numbered relative to the full length wild-type PTH(1-17). Thus, for example, a reference to position 11 should be construed as a reference to the 11th residue from the N-terminus of PTH(1-17). In this connection, it should be noted that in embodiments of the invention where the amino acid at position 16 is absent, the C-terminal amino acid is still defined as position 17.

In particular, the present application relates to PTH(1-17) peptides which have one or more substitutions relative to wild-type PTH(1-17), and which may have improved properties as compared to wild type PTH(1-17) and [Ac₅c¹, Aib³]MPTH(1-14). These substitutions may comprise a conservative substitution at any amino acid position optionally in combination with at least one non-conservative amino acid substitution. In particular, the present invention relates to a cyclic link between residue A13 and residue A17, e.g. a cyclic link formed between the side chains of the amino acid residues at these positions.

Accordingly, in one aspect, the present invention relates to a biologically active PTH(1-17)analogue peptide represented by Formula I which consists of:

### R1-Z1-A1-A2-A3-A4-A5-A6-A7-A8-A9-A10-A11-A12-A13-A14-Leu-A16-A17-Z2-R2

wherein
R1 is hydrogen, NH₂, RHN, RR3N, wherein each of R and R3 independently represent C1-4 alkyl (e.g. methyl), acetyl, formyl, benzoyl or trifluoroacetyl;
A1 is Ac5c, Gly, Ser, Ala or any alpha-helix stabilizing residue;
A2 is Val or a conservative substitution;
A3 is Aib, Ala, Ser or any alpha-helix stabilizing residue;
A4 is Glu or a conservative substitution;
A5 is Ile or a conservative substitution;
A6 is Gln, Glu or a conservative substitution;
A7 is Leu or Phe or a conservative substitution;
A8 is Met, Leu, Nle, Val or a conservative substitution;
A9 is His or a conservative substitution;
A10 is Gln, Glu, Asp, Ala, Val or a conservative substitution;
A11 is Har, Arg, Ala, Ile, Lys or a conservative substitution;
A12 is Ala, Arg, His or a conservative substitution;

### A13 is Lys, Orn, Asp, Glu, Cys, Dab or Dpr;

A14 is Trp, Phe, Leu, Arg, His or a conservative substitution;
A16 is Asn, Asp, a conservative substitution or absent;

### A17 is, Lys, Orn, Glu, Cys, Asp, Dab or Dpr; and

R2 is OH, OR, NRH, NRR3 or NH₂, wherein each of R and R3 independently represents C1-4 alkyl (e.g. methyl); and
A13 and A17 are linked by one or more covalent bonds; and
Z1 and Z2 are independently absent, or a peptide sequence of 1-10 amino acid units selected from the group consisting of Ala, Leu, Met, Gln, Glu, Lys, Dab, Dpr and Orn;
or a homodimer, heterodimer, or a pharmaceutically acceptable salt or derivative thereof.

As is well known in the art, alpha-helix stabilizing residues include Gly, Ser and Ala, as well as non natural amino acid residues such as Ac5c, Ac6c, Abu, Nva and Aib.

In a further aspect, the present invention provides a biologically active PTH(1-17) analogue peptide represented by Formula II which consists of:

### R1-Z1-A1-Val-A3-Glu-Ile-A6-A7-A8-His-A10-A11-A12-A13-A14-Leu-A16-A17-Z2-R2

wherein
R1 is hydrogen, NH₂, RHN, RR3N, wherein each of R and R3 independently represent C1-4 alkyl (e.g. methyl), acetyl, formyl, benzoyl or trifluoroacetyl;
A1 is Ac5c, Gly, Ser, Ala or any alpha-helix stabilizing residue;
A3 is Aib, Ala, Ser or any alpha-helix stabilizing residue;

### A6 is Gln or Glu;

### A7 is Leu or Phe;

### A8 is Met, Leu, Nle or Val;

### A10 is Gln, Glu, Asp, Ala or Val;

### A11 is Har, Arg, Ala, Ile or Lys;

### A12 is Ala, Arg or His;

### A13 is Lys, Orn, Asp, Glu, Cys, Dab or Dpr;

### A14 is Trp, Phe, Leu, Arg or His;

A16 is Asn, Asp or absent;

### A17 is Lys, Orn, Glu, Cys, Asp, Dab or Dpr;

R2 is OH, OR, NRH, NRR3 or NH₂, wherein each of R and R3 independently represents C1-4 alkyl (e.g. methyl); and
A13 and A17 are linked by one or more covalent bonds; and
Z1 and Z2 are independently absent, or a peptide sequence of 1-10 amino acid units selected from the group consisting of Ala, Leu, Lys, Dab, Dpr and Orn;
or a homodimer, heterodimer, or pharmaceutically acceptable salt or derivative thereof.

In a further aspect, the present invention provides a substituted PTH(1-17) analogue peptide having the Formula III:

R1-Z1-Ac5c-Val-Aib-Glu-Ile-A6-Leu-A8-His-A10-A11-Ala-A13-A14-Leu-A16-A17-Z2-R2
wherein
R1 is hydrogen, NH₂, RHN, RR3N, wherein each of R and R3 independently represent C1-4 alkyl (e.g. methyl), acetyl, formyl, benzoyl or trifluoroacetyl;

### A6 is Glu or Gln;

### A8 is Met, Leu, Nle or Val;

### A10 is Gln or Glu;

### A11 is Har or Arg;

### A13 is Lys, Orn, Asp, Glu, Cys, Dab or Dpr;

### A14 is Trp or Phe;

A16 is Asn, Asp or absent;

### A17 is Lys, Orn, Glu, Cys, Asp, Dab or Dpr;

R2 is OH, OR, NRH, NRR3 or NH₂, wherein each of R and R3 independently represents C1-4 alkyl (e.g. methyl); and
A13 and A17 are linked by one or more covalent bonds; and
Z1 and Z2 are independently absent, or a peptide sequence of 1-10 amino acid units selected from the group consisting of Ala, Leu, Glu, Lys, Dab, Dpr and Orn,
or a homodimer, heterodimer or pharmaceutically acceptable salt or derivative thereof.

In a further aspect, the present invention provides a biologically active PTH(1-17) analogue peptide represented by Formula IV which consists of:

### R1-Z1-A1-Val-A3-Glu-Ile-A6-A7-A8-His-A10-A11-A12-A13-A14-Leu-A16-A17-Z2-R2

wherein
R1 is hydrogen, NH₂, RHN, RR3N, wherein each of R and R3 independently represent C1-4 alkyl (e.g. methyl), acetyl, formyl, benzoyl or trifluoroacetyl;

### A1 is Ac5c, Ac6c, Abu, Nva or Aib;

### A3 is Ac5c, Aib, Abu or Nva;

### A6 is Gln or Glu;

### A7 is Leu or Phe;

### A8 is Met, Leu, Val or Nle;

### A10 is Gln or Glu;

### A11 is Har or Arg;

### A12 is Ala or Arg;

### A13 is Lys, Glu, Asp or Cys;

### A14 is Trp or Phe;

A16 is Asn, Asp or absent;

### A17 is, Glu, Cys, Asp or Lys;

R2 is OH, OR, NRH, NRR3 or NH₂, wherein each of R and R3 independently represents C1-4 alkyl (e.g. methyl); and
A13 and A17 are linked by one or more covalent bonds; and
Z1 and Z2 are independently absent, or a peptide sequence of 1-10 amino acid units selected from the group consisting of Ala, Leu, Lys, Dab, Dpr and Orn;
or a homodimer, heterodimer, or pharmaceutically acceptable salt or derivative thereof.

In a further aspect, the present invention provides a biologically active PTH(1-17) analogue peptide represented by Formula V which consists of:

### R1-Z1-A1-Val-Aib-Glu-Ile-Gln-A7-A8-His-Gln-A11-A12-A13-Trp-Leu-A16-A17-Z2-R2

wherein
R1 is hydrogen, NH₂, RHN, RR3N, wherein each of R and R3 independently represent C1-4 alkyl (e.g. methyl), acetyl, formyl, benzoyl or trifluoroacetyl;

### A1 is Ac5c or Ac6c;

### A7 is Leu or Phe;

### A8 is Met, Leu or Nle;

### A11 is Har or Arg;

### A12 is Ala or Arg;

### A13 is Lys or Glu;

A16 is Asn or absent;

### A17 is, Glu, or Asp;

R2 is OH, OR, NRH, NRR3 or NH₂, wherein each of R and R3 independently represents C1-4 alkyl (e.g. methyl); and
A13 and A17 are linked by one or more covalent bonds; and
Z1 and Z2 are independently absent, or a peptide sequence of 1-10 amino acid units selected from the group consisting of Ala, Leu, Lys, Dab, Dpr and Orn;
or a homodimer, heterodimer, or pharmaceutically acceptable salt or derivative thereof.

There are many possibilities for side-chain-to-side-chain cyclisations or bridges, including but not limited to, amides (lactams), esters (lactones), ethers, ketones or disulfides (Synthetic Peptides, A users guide. 2nd ed. 2002. Oxford University Press. Ed. Grant, G. A). Any of these possibilities may be used to covalently link the side chains of the A13 and A17 amino acid residues in the formulae defined above.

In a particularly preferred embodiment, the covalent bonding between A13 and A17 comprises a lactam bridge or a cysteine bridge.

In another embodiment of the present invention, the PTH analogue is provided in the form of a dimer. The dimer may be formed as a homodimer of a PTH analogue such as, but not limited to, Acsc-Val-Aib-Glu-Ile-Gln-Leu-Met-His-Gln-Har-Ala-Lys-Trp-Leu-Asn-Asp-NH₂.

In another embodiment of the present invention, the dimer formed is a heterodimer of two different PTH analogues such as, but not limited to, Ac₅c-Val-Aib-Glu-Ile-Gln-Leu-Met-His-Gln-Har-Ala-Lys-Trp-Leu-Asn-Asp-NH₂ and Ac₅c-Val-Aib-Glu-Ile-Gln-Leu-Met-His-Gln-Har-Ala-Lys-Trp-Leu-Asp-NH₂.

In a further aspect, the present invention relates to a substituted PTH(1-17) peptide which comprises at least two and up to 14 substitutions relative to wild type human PTH(1-17) between residues A1 and A17 inclusive.

The peptides of Formula I, II or III preferably comprises 1 or 2 substitutions at positions 1 or 3 relative to wild-type PTH, optionally in combination with 1, 2, 3, 4, 5, 6, 7, 8, 9 or 10 substitutions at further positions, including position 6, 7, 8, 10, 11, 12, 13, 14, 16 or 17.

Examples of combinations of residues at positions 6, 7, 8, 10, 11, 12, 13, 14, 16 or 17 which may be present in the analogues of the invention, and fall within Formulae I to III:

| | |
|---|---|
| H-Ac₅c-Val-Aib-Glu-Ile-Gln-Leu-Met-His-Gln-Har-Ala-Lys( )Trp-Leu-Asn-Asp( )-NH₂ | (SEQ ID NO: 2) |
| H-Ac₅c-Val-Aib-Glu-Ile-Gln-Leu-Met-His-Gln-Har-Ala-Lys( )Trp-Leu-Asn-Asp( )-OH | (SEQ ID NO: 4) |
| H-Ac₅c-Val-Aib-Glu-Ile-Gln-Leu-Leu-His-Gln-Har-Ala-Lys( )Trp-Leu-Asn-Asp( )-OH | (SEQ ID NO: 5) |
| H-Ac₅c-Val-Aib-Glu-Ile-Gln-Leu-Met-His-Gln-Har-Ala-Asp( )Trp-Leu-Asn-Lys( )-NH₂ | (SEQ ID NO: 6) |
| H-Ac₅c-Val-Aib-Glu-Ile-Gln-Leu-Met-His-Gln-Har-Ala-Cys( )Trp-Leu-Asn-Cys( )-NH₂ | (SEQ ID NO: 7) |
| H-Ac₅c-Val-Aib-Glu-Ile-Gln-Leu-Met-His-Gln-Har-Ala-Glu( )Trp-Leu-Asn-Lys( )-NH₂ | (SEQ ID NO: 8) |
| H-Ac₅c-Val-Aib-Glu-Ile-Gln-Leu-Met-His-Gln-Har-Ala-Lys( )Trp-Leu-Asn-Glu( )-NH₂ | (SEQ ID NO: 9) |
| H-Ac₆c-Val-Aib-Glu-Ile-Gln-Leu-Leu-His-Gln-Har-Ala-Lys( )Trp-Leu-Asn-Asp( )-NH₂ | (SEQ ID NO: 10) |
| H-Abu-Val-Aib-Glu-Ile-Gln-Leu-Leu-His-Gln-Har-Ala-Lys( )Trp-Leu-Asn-Asp( )-NH₂ | (SEQ ID NO: 11) |
| H-Nva-Val-Aib-Glu-Ile-Gln-Leu-Leu-His-Gln-Har-Ala-Lys( )Trp-Leu-Asn-Asp( )-NH₂ | (SEQ ID NO: 12) |
| H-Aib-Val-Ac₅c-Glu-Ile-Gln-Leu-Leu-His-Gln-Har-Ala-Lys( )Trp-Leu-Asn-Asp( )-NH₂ | (SEQ ID NO: 13) |
| H-Ac₅c-Val-Ac₅c-Glu-Ile-Gln-Leu-Leu-His-Gln-Har-Ala-Lys( )Trp-Leu-Asn-Asp( )-NH₂ | (SEQ ID NO: 14) |
| H-Ac₅c-Val-Abu-Glu-Ile-Gln-Leu-Leu-His-Gln-Har-Ala-Lys( )Trp-Leu-Asn-Asp( )-NH₂ | (SEQ ID NO: 15) |
| H-Ac₅c-Val-Nva-Glu-Ile-Gln-Leu-Leu-His-Gln-Har-Ala-Lys( )Trp-Leu-Asn-Asp( )-NH₂ | (SEQ ID NO: 16) |
| H-Ac₅c-Val-Aib-Glu-Ile-Glu-Leu-Met-His-Gln-Har-Ala-Lys( )Trp-Leu-Asn-Asp( )-NH₂ | (SEQ ID NO: 18) |
| H-Ac₅c-Val-Aib-Glu-Ile-Gln-Leu-Leu-His-Gln-Har-Ala-Lys( )Trp-Leu-Asn-Asp( )-NH₂ | (SEQ ID NO: 19) |
| H-Ac₅c-Val-Aib-Glu-Ile-Gln-Leu-Nle-His-Gln-Har-Ala-Lys( )Trp-Leu-Asn-Asp( )-NH₂ | (SEQ ID NO: 20) |
| H-Ac₅c-Val-Aib-Glu-Ile-Gln-Leu-Val-His-Gln-Har-Ala-Lys( )Trp-Leu-Asn-Asp( )-NH₂ | (SEQ ID NO: 21) |
| H-Ac₅c-Val-Aib-Glu-Ile-Gln-Leu-Leu-His-Gln-Har-Ala-Lys( )Trp-Leu-Asp-Asp( )-NH₂ | (SEQ ID NO: 22) |
| H-Ac₅c-Val-Aib-Glu-Ile-Gln-Leu-Met-His-Gln-Har-Ala-Lys( )Trp-Leu-Asp-Asp( )-NH₂ | (SEQ ID NO: 23) |
| H-Ac₅c-Val-Aib-Glu-Ile-Gln-Leu-Met-His-Glu-Har-Ala-Lys( )Trp-Leu-Asn-Asp( )-NH₂ | (SEQ ID NO: 24) |
| H-Ac₅c-Val-Aib-Glu-Ile-Gln-Leu-Met-His-Gln-Arg-Ala-Lys( )Trp-Leu-Asn-Asp( )-NH₂ | (SEQ ID NO: 25) |
| H-Ac₅c-Val-Aib-Glu-Ile-Gln-Leu-Met-His-Gln-Har-Ala-Lys( )Phe-Leu-Asn-Asp( )-NH₂ | (SEQ ID NO: 26) |
| H-Ac₅c-Val-Aib-Glu-Ile-Gln-Phe-Leu-His-Gln-Har-Ala-Lys( )Trp-Leu-Asn-Asp( )-NH₂ | (SEQ ID NO: 27) |
| H-Ac₅c-Val-Aib-Glu-Ile-Gln-Leu-Leu-His-Gln-Har-Arg-Lys( )Trp-Leu-Asn-Asp( )-NH₂ | (SEQ ID NO: 28) |
| H-Ac₅c-Val-Aib-Glu-Ile-Glu-Leu-Leu-His-Gln-Har-Ala-Lys( )Trp-Leu-Asn-Asp( )-NH₂ | (SEQ ID NO: 29) |
| H-Ac₅c-Val-Aib-Glu-Ile-Gln-Leu-Met-His-Gln-Har-Ala-Lys( )Trp-Leu-Asp( )-NH₂ | (SEQ ID NO: 30) |
| H-Ac₅c-Val-Aib-Glu-Ile-Gln-Leu-Met-His-Gln-Har-Ala-Lys-Trp-NH₂ | (SEQ ID NO: 31) |
| (H-Ac₅c-Val-Aib-Glu-Ile-Gln-Leu-Met-His-Gln-Har-Ala-Lys( )-Trp-Leu-Asp( )-NH₂₎₂ | (SEQ ID NO: 33) |
| (H-Ac₅c-Val-Aib-Glu-lle-Gln-Leu-Met-His-Gln-Har-Ala-Lys( )-Trp-Leu-Asn-Asp( )-NH₂)₂ | (SEQ ID NO: 39) |
| H-Ac₅c-Val-Aib-Glu-Ile-Gln-Leu-Met(O)-His-Gln-Har-Ala-Lys( )Trp-Leu-Asn-Asp( )-NH₂ | (SEQ ID NO: 40) |
| H- Ac₅c-Val-Aib-Glu-Ile-Gln-Leu-Leu-His-Gln-Har-Ala-Dab( )Trp-Leu-Asn-Asp( )-NH₂ | (SEQ ID NO: 41) |
| where brackets ( ) indicate cyclisation sites. | |

Conservative substitutions in the peptides of the invention are grouped in five Groups I to V as shown in Table 1 below where the one-letter code for natural amino acids are used:

TABLE 1
Conservative substitutions of amino acids
grouped by physicochemical properties.

| I | II | III | IV | V |
|---|---|---|---|---|
| A | N | H | M | F |
| S | D | R | L | Y |
| T | E | K | I | W |
| P | Q | | V | |
| G | | | C | |

I: neutral, hydrophilic, II: acids and amides, III: basic, IV: hydrophobic, V: aromatic, bulky amino acids.

However, in other embodiments of the invention, Z1 and/or Z2 may be absent.

Particular peptide sequences falling within the scope of Formulae I to III are set forth in Table 3.

Most particular, the present invention is particularly concerned with PTH analogues in which there is a covalent link between A13 and A17 of the PTH(1-17) analogues. As shown herein, the covalent bond between A13 and A17 of the PTH(1-17) analogues has a profound effect on the potency of said peptides as compared to the similar PTH agonist with no covalent bond.

In a further aspect, the present invention provides a method of medical treatment, comprising administering to a subject in need of treatment a PTH peptide as defined herein.

In a further aspect, the present invention provides a PTH peptide of the invention for use in therapy.

In a further aspect, the present invention also provides pharmaceutical compositions comprising a PTH derivative and a pharmaceutically acceptable excipient and/or a pharmaceutically acceptable solution such as saline or a physiologically buffered solution.

In a further aspect, the present invention also provides a method for treating mammalian conditions characterized by decreases in bone mass, which method comprises administering to a subject in need thereof an effective bone mass-increasing amount of a biologically active PTH polypeptide. A preferable embodiment of the invention is drawn to conditions such as osteoporosis. The types of osteoporosis include, but are not limited to old age osteoporosis and postmenopausal osteoporosis.

In a further aspect, the present invention provides a method of increasing cAMP in a mammalian cell having PTH-1 receptors, the method comprising contacting the cell with a sufficient amount of the polypeptide of the invention to increase cAMP.

Further, these polypeptide analogues are useful in the treatment of patients with bone loss. Bone loss may result from conditions such as osteoporosis, glucocorticoid-induced bone loss, hypercortisolism (both subclinical and clinical), cancer, hypercalcemia, renal failure or other kidney disorders, renal transplant and accompanying pharmacological treatments, cholestatic liver diseases, viral hepatitis, bone loss caused by liver transplant, hyperparathyroid disease, bronchial asthma (including hormone-dependent), disorders due to haemodialysis, and osteomalacia.

As shown in the examples, the presence of the cyclic covalently bonded structure of the peptides of the present invention preferably has the advantage that it helps to prevent inhibition of cytochrome P450 enzymes, and in particular CYP2D6, that is observed with linear PTH(1-17) analogues. Alternatively or additionally, as shown herein, the cyclised analogues of the present invention provide an increase in bone mineral density and/or bone strength in the ovariectomized (OVX) rat model significantly above sham level, and which have not been previously observed with linear PTH(1-17) analogues.

Thus, the fact that SEQ ID NO: 19 and other semi-cyclic analogues has preserved activity on the PTH receptor and stimulates bone formation in absence of any inhibitory effect on CYP2D6 activity, indicate that prolonged treatment with this compound does not affect the pharmacokinetics of other drugs or herbal products metabolized by this enzyme. Therefore, we expect that long-term treatment with this novel class of compounds will be associated with increased safety. Furthermore, in the elderly population who are often taken multiple drugs and herbal supplements this feature may be particularly important. Due to little available information on herb-drug interactions, these are often misinterpreted as poor tolerability of the drug. Therefore, the lack of effects of CYP2D6 activity could potentially be important for compliance to the prescribed drug, which in turn may provide into better long-term efficacy.

Embodiments of the present invention will now be described in more detail, by way of example and not limitation, with reference to the accompanying figures.

### BRIEF DESCRIPTION OF THE FIGURES

FIG. 1: Representative cAMP efficacy experiment on MC3T3-E1 cells with novel PTH(1-17) analogues. Cells were stimulated with varying concentrations of PTH peptides for 15 min at 37° C. in the presence of a phosphodiesterase inhibitor. Error bars show standard deviation of triplicates.

FIG. 2: Representative cAMP efficacy experiment on MC3T3-E1 cells with novel cyclic monomer SEQ ID NO: 30 and the corresponding covalent homodimer SEQ ID NO: 33. Cells were stimulated with varying concentrations of PTH peptides for 15 min at 37° C. in the presence of a phosphodiesterase inhibitor. Error bars show standard deviation of triplicates.

FIG. 3: cAMP-efficacy assays with PTH(1-17) analogues on Saos-2 cells. The cyclic PTH-analogue SEQ ID NO:2 showed significantly higher potency than non-cyclic analogues containing the same α-helix-stabilizing amino acid residues. Error bars designate the standard error of the mean.

FIG. 4: cAMP-efficacy assays with PTH(1-17) analogues on Saos-2 cells. The cyclic PTH-analogue SEQ ID NO:4 showed higher potency than a non-cyclic analogue containing the same α-helix-stabilizing amino acid residues. Data shown are combined from two separate experiments with similar maximal effect. Error bars designate the standard error of the mean.

FIG. 5: cAMP-efficacy assays with PTH(1-17) analogues on Saos-2 cells. The cyclic PTH-analogue SEQ ID NO:34 also containing α-helix-stabilizing amino acids showed strikingly higher potency than non-cyclic analogues with or without the same α-helix-stabilizing amino acid residues and a cyclic analogue devoid of α-helix-stabilizing amino acids. Determinations were done as single determinations.

FIG. 6: Determination of bone mineral density (BMD) by DEXA scanning. The 18% bone loss in the proximal tibia of OVX animals, representing a highly responsive site rich in trabecular bone, was completely reversed by SEQ ID NO:19 and SEQ ID NO:33. Error bars designate the standard error of the mean.

FIG. 7: Determination of bone mineral density (BMD) by DEXA scanning. BMD in lumbar vertebrae L₁-L₂ was improved by SEQ ID NO: 19 and SEQ ID NO:33 relative to BMD in vehicle-treated OVX and sham animals. Error bars designate the standard error of the mean.

FIG. 8: Determination of bone mineral density (BMD) by DEXA scanning. BMD in SEQ ID NO:19 and SEQ ID NO:33-treated animals was significantly increased compared to vehicle-treated OVX animals at all doses, and was significantly increased over sham control levels at doses higher than 20 nmol/kg/d. Error bars designate the standard error of the mean.

FIG. 9: Determination of bone mineral density (BMD) by DEXA scanning. BMD in the femoral shaft, a site rich in cortical bone, was improved by SEQ ID NO:19 and SEQ ID NO:33. An about 81 increase in BMD was noted compared to vehicle-treated OVX animals. Error bars designate the standard error of the mean.

FIG. 10: Bone strength measurements. Bone strength of the femoral head in a compression test was improved by SEQ ID NO:19 and SEQ ID NO:33. Bone strength was higher than in vehicle-treated OVX and sham animals at all doses. Error bars designate the standard error of the mean.

FIG. 11: Bone strength measurements. Bone strength of the femur in a three-point bending test was improved by SEQ ID NO:19 and SEQ ID NO:33. Bone strength was higher than in vehicle-treated OVX animals at all doses, and doses of SEQ ID NO:19 higher than 20 nmol/kg/d even led to increased strength compared to vehicle-treated sham animals. Error bars designate the standard error of the mean.

### DETAILED DESCRIPTION

### Definitions

Throughout the description and claims the conventional one-letter and three-letter code for natural amino acids are used, as well as generally accepted three letter codes for other α-amino acids, such as norleucine (Nle), homoarginine (Har), 1-aminocyclopentanecarboxylic acid (Ac₅c), 2,4-diaminobytyric acid (Dab), 2,3-diaminopropionic acid (Dpr), 2,5-diaminopentanonic acid (Orn) and α-amino isobutanoic acid (Aib).

The PTH analogues of the invention contain residues which are described as being positively or negatively charged. This should be understood to mean that the side chain functionalities of the residues in question carry a whole or partial positive or negative charge at physiological pH, which is considered to be approximately 7.4.

It will be understood that a single residue cannot carry a partial positive charge. This term instead refers to the average charge on the relevant residue over the whole population of peptides having the same sequence in a given system. This will be between 0 and 1 if the pK of the ionisable side chain functionality of the residue in question is within about 2 pH units of 7.4; i.e. between about 5.4 and about 9.4.

The pKₐ of a “positively charged” residue is preferably above about 6. The pKₐ of a “negatively charged” residue is preferably below about 8. Examples of “positively charged” residues include Lys, Arg, Har, His, Orn, Dab and Dpr.

Examples of “negatively charged” residues include Asp and Glu.

“Neutral” residues are those which carry substantially no charge at physiological pH. These include Gln, Asn, Ala, Gly, Ser, Thr, Ile, Leu, Met, Phe, Pro, Trp, Val.

“Aromatic” residues include His, Phe, Tyr and Trp.

Conservative substitutions in the peptides of the invention are grouped in five groups I to V as shown in Table 2 below where the one-letter code for natural amino acids are used:

TABLE 2
Conservative substitutions of amino acids
grouped by physicochemical properties.

| I | II | III | IV | V |
|---|---|---|---|---|
| A | N | H | M | F |
| S | D | R | L | Y |
| T | E | K | I | W |
| P | Q | | V | |
| G | | | C | |

I: neutral, hydrophilic, II: acids and amides, III: basic, IV: hydrophobic, V: aromatic, bulky amino acids.

The amino acid residues of the invention may have either D- or L-configuration, but preferably they have an L-configuration.

For reference, PTH is secreted as an 84 amino acid peptide with the following sequence H-Ser-Val-Ser-Glu-Ile-Gln-Leu-Met-His-Asn-Leu-Gly-Lys-His-Leu-Asn-Ser-Met-Glu-Arg-Val-Glu-Trp-Leu-Arg-Lys-Lys-Leu-Gln-Asp-Val-His-Asn-Phe-Val-Ala-Leu-Gly-Ala-Pro-Leu-Ala-Pro-Arg-Asp-Ala-Gly-Ser-Gln-Arg-Pro-Arg-Lys-Lys-Glu-Asp-Asn-Val-Leu-Val-Glu-Ser-His-Glu-Lys-Ser-Leu-Gly-Glu-Ala-Asp-Lys-Ala-Asp-Val-Asn-Val-Leu-Thr-Lys-Ala-Lys-Ser-Gln-OH.

The PTH analogues of the present invention have one or more amino acid substitutions, deletions, or additions compared with native PTH as defined above.

Surprisingly, substituted PTH(1-17) analogue molecules have been found to exhibit cAMP accumulation sustained activity towards PTH receptors, such as PTH-1 receptors, and which are also active in vivo, as illustrated in the examples below.

In another aspect, the present invention provides novel peptides with both improved chemical and pharmaceutical stability against degradation compared to PTH(1-17) and [Ac₅c¹, Aib³]MPTH(1-14).

Modification at one or more of positions 6, 8, 10, 11, 13, 14, 16 or 17 by substitution with Ala, Leu, Nle, Val, Ser, Glu, Asp, Lys or Arg of [Ac₅c¹, Aib³]MPTH(1-14) increases the chemical stability of the molecule and may thus improve shelf-life and reduce degradation during formulation.

The analogue of the present invention may include chemical modification of one or more of its amino acid side chain functionalities, terminal amino group, or terminal carboxylic acid group. A chemical modification includes, but is not limited to, adding chemical moieties, creating new bonds, and removing chemical moieties. Modifications at amino acid side groups include, without limitation, acylation of lysine epsilon-amino groups, N-alkylation of arginine, histidine, or lysine, esterification of glutamic or aspartic carboxylic acid groups, and deamidation of glutamine or asparagine. Modifications of the terminal amino include, without limitation, the des-amino, N-lower alkyl, N-di-lower alkyl, and N-acyl modifications. Modifications of the terminal carboxy group include, without limitation, the amide, lower alkyl amide, dialkyl amide, and lower alkyl ester modifications. Preferably herein lower alkyl is C1-C4 alkyl. Furthermore, one or more side groups; or terminal groups, may be protected by protective groups known to the ordinarily-skilled peptide chemist.

As used herein, “biological activity” refers to the bone-anabolic activity of PTH-analogues or derivatives thereof, that leads to the formation of mineralized bone in adult vertebrates, as demonstrated in the examples using the OVX rat experimental model. Preferably, this biological activity is determined at appropriate doses in an intermittent dosing regimen.

Alternatively or additionally, a further biological activity of the PTH peptides of the present invention is that they do not significantly inhibit the activity of a cytochrome P450 (CYP) enzyme. By way of example, preferably means that the activity, measured as the formation rate of a CYP isoform specific metabolite, is not reduced more than 30%, and preferably not more than 20%, by the PTH peptide of the present invention as compared to a control, e.g. the formation rate in presence of vehicle alone.

It should be understood that the peptides of the invention might also be provided in the form of a salt or other derivative. Salts include pharmaceutically acceptable salts such as acid addition salts and basic salts. Examples of acid addition salts include hydrochloride salts, citrate salts and acetate salts. Examples of basic salts include salts where the cation is selected from alkali metals, such as sodium and potassium, alkaline earth metals, such as calcium, and ammonium ions +N (R3)3 or (R4) where R3 and R4 independently designates optionally substituted C(1-6)-alkyl, optionally substituted C(2-6)-alkenyl, optionally substituted aryl, or optionally substituted heteroaryl.

Other examples of pharmaceutically acceptable salts are described in “Remington's Pharmaceutical Sciences”, Mack Publishing Company, Easton, Pa., 19th Edition, 1995, and in the Encyclopaedia of Pharmaceutical Technology.

Other derivatives of the PTH analogues of the invention include coordination complexes with metal ions such as Mn2+ and Zn2+, esters such as in vivo hydrolysable esters, free acids or bases, hydrates, prodrugs or lipids. Esters can be formed between hydroxyl or carboxylic acid groups present in the compound and an appropriate carboxylic acid or alcohol reaction partner, using techniques well known in the art. Derivatives which acts as prodrugs of the compounds are convertible in vivo or in vitro into one of the parent compounds. Typically, at least one of the biological activities of compound will be reduced in the prodrug form of the compound, and can be activated by conversion of the prodrug to release the compound or a metabolite of it. Examples of prodrugs include the use of protecting groups which may be removed in situ releasing active compound or serve to inhibit clearance of the drug in vivo.

In certain embodiments of the present invention, Z1 and Z2 are peptide sequence of 1-10 amino acid residues, e.g., in the range of 2-8 in particular in the range of 3-6 amino acid residues, e.g., of 2, 3, 4, 5 or 6 amino acid residues. Typically, only one of Z1 and Z2 is present, such as Z1. Each of the amino acid residues in the peptide sequence Z are independently selected from Ala, Leu, Ser, Thr, Tyr, Asn, Gln, Asp, Glu, Lys, Arg, His, Orn. Preferably, the amino acid residues are selected from Ser, Thr, Tyr, Asn, Gln, Asp, Lys, Arg, His, Orn, Dab and Dpr, especially Lys. The above-mentioned amino acids may have either D- or L-configuration, but preferably the above-mentioned amino acids have an L-configuration.

Such peptides at the N and/or C-terminus of the molecule are believed to increase solubility of the PTH analogue peptides and increase stability, e.g. against protease activity, thus leading to improved pharmacokinetic properties, such as increased half life and reduced tendency to aggregate.

Examples of PTH peptides are shown in Table 3 below. Some of the peptides are controls and are provided by way of comparison with the peptides of the present invention (e.g., see PTH1-34, SEQ ID NO: 32). A preferred group of peptides of the present invention are shown in bold text in the table.

TABLE 3
Peptides of relevance

| | |
|---|---|
| H-Ac₅c-Val-Aib-Glu-Ile-Gln-Leu-Met-His-Asn-Leu-Gly-Lys-His-Leu-Asn-Asp-NH₂ | (SEQ ID NO: 1) |
| H-Ac₅c-Val-Aib-Glu-Ile-Gln-Leu-Met-His-Gln-Har-Ala-Lys( )Trp-Leu-Asn-Asp( )-NH₂ | (SEQ ID NO: 2) |
| H-Ac₅c-Val-Aib-Glu-Ile-Gln-Leu-Met-His-Gln-Har-Ala-Lys-Trp-Leu-Asn-Asn-NH₂ | (SEQ ID NO: 3) |
| H-Ac₅c-Val-Aib-Glu-Ile-Gln-Leu-Met-His-Gln-Har-Ala-Lys( )-Trp-Leu-Asn-Asp( )-OH | (SEQ ID NO: 4) |
| H-Ac₅c-Val-Aib-Glu-Ile-Gln-Leu-Leu-His-Gln-Har-Ala-Lys( )-Trp-Leu-Asn-Asp( )-OH | (SEQ ID NO: 5) |
| H-Ac₅c-Val-Aib-Glu-Ile-Gln-Leu-Met-His-Gln-Har-Ala-Asp( )-Trp-Leu-Asn-Lys( )-NH₂ | (SEQ ID NO: 6) |
| H-Ac₅c-Val-Aib-Glu-Ile-Gln-Leu-Met-His-Gln-Har-Ala-Cys( )-Trp-Leu-Asn-Cys( )-NH₂ | (SEQ ID NO: 7) |
| H-Ac₅c-Val-Aib-Glu-Ile-Gln-Leu-Met-His-Gln-Har-Ala-Glu( )-Trp-Leu-Asn-Lys( )-NH₂ | (SEQ ID NO: 8) |
| H-Ac₅c-Val-Aib-Glu-Ile-Gln-Leu-Met-His-Gln-Har-Ala-Lys( )-Trp-Leu-Asn-Glu( )-NH₂ | (SEQ ID NO: 9) |
| H-Ac₆c-Val-Aib-Glu-Ile-Gln-Leu-Leu-His-Gln-Har-Ala-Lys( )-Trp-Leu-Asn-Asp( )-NH₂ | (SEQ ID NO: 10) |
| H-Abu-Val-Aib-Glu-Ile-Gln-Leu-Leu-His-Gln-Har-Ala-Lys( )-Trp-Leu-Asn-Asp( )-NH₂ | (SEQ ID NO: 11) |
| H-Nva-Val-Aib-Glu-Ile-Gln-Leu-Leu-His-Gln-Har-Ala-Lys( )-Trp-Leu-Asn-Asp( )-NH₂ | (SEQ ID NO: 12) |
| H-Aib-Val-Ac₅c-Glu-Ile-Gln-Leu-Leu-His-Gln-Har-Ala-Lys( )-Trp-Leu-Asn-Asp( )-NH₂ | (SEQ ID NO: 13) |
| H-Ac₅c-Val-Ac₅c-Glu-Ile-Gln-Leu-Leu-His-Gln-Har-Ala-Lys( )-Trp-Leu-Asn-Asp( )-NH₂ | (SEQ ID NO: 14) |
| H-Ac₅c-Val-Abu-Glu-Ile-Gln-Leu-Leu-His-Gln-Har-Ala-Lys( )-Trp-Leu-Asn-Asp( )-NH₂ | (SEQ ID NO: 15) |
| H-Ac₅c-Val-Nva-Glu-Ile-Gln-Leu-Leu-His-Gln-Har-Ala-Lys( )-Trp-Leu-Asn-Asp( )-NH₂ | (SEQ ID NO: 16) |
| H-Ac₅c-Val-Aib-Glu-Ile-Gln-Leu-Leu-His-Gln-Har-Ala-Dpr( )-Trp-Leu-Asn-Asp( )-NH₂ | (SEQ ID NO: 17) |
| H-Ac₅c-Val-Aib-Glu-IIe-Glu-Leu-Met-His-Gln-Har-Ala-Lys( )-Trp-Leu-Asn-Asp( )-NH₂ | (SEQ ID NO: 18) |
| H-Ac₅c-Val-Aib-Glu-Ile-Gln-Leu-Leu-His-Gln-Har-Ala-Lys( )-Trp-Leu-Asn-Asp( )-NH₂ | (SEQ ID NO: 19) |
| H-Ac₅c-Val-Aib-Glu-Ile-Gln-Leu-Nle-His-Gln-Har-Ala-Lys( )-Trp-Leu-Asn-Asp( )-NH₂ | (SEQ ID NO: 20) |
| H-Ac₅c-Val-Aib-Glu-Ile-Gln-Leu-Val-His-Gln-Har-Ala-Lys( )-Trp-Leu-Asn-Asp( )-NH₂ | (SEQ ID NO: 21) |
| H-Ac₅c-Val-Aib-Glu-Ile-Gln-Leu-Leu-His-Gln-Har-Ala-Lys( )-Trp-Leu-Asp-Asp( )-NH₂ | (SEQ ID NO: 22) |
| H-Ac₅c-Val-Aib-Glu-Ile-Gln-Leu-Met-His-Gln-Har-Ala-Lys( )-Trp-Leu-Asp-Asp( )-NH₂ | (SEQ ID NO: 23) |
| H-Ac₅c-Val-Aib-Glu-Ile-Gln-Leu-Met-His-Glu-Har-Ala-Lys( )-Trp-Leu-Asn-Asp( )-NH₂ | (SEQ ID NO: 24) |
| H-Ac₅c-Val-Aib-Glu-Ile-Gln-Leu-Met-His-Gln-Arg-Ala-Lys( )-Trp-Leu-Asn-Asp( )-NH₂ | (SEQ ID NO: 25) |
| H-Ac₅c-Val-Aib-Glu-Ile-Gln-Leu-Met-His-Gln-Har-Ala-Lys( )-Phe-Leu-Asn-Asp( )-NH₂ | (SEQ ID NO: 26) |
| H-Ac₅c-Val-Aib-Glu-Ile-Gln-Phe-Leu-His-Gln-Har-Ala-Lys( )-Trp-Leu-Asn-Asp( )-NH₂ | (SEQ ID NO: 27) |
| H-Ac₅c-Val-Aib-Glu-Ile-Gln-Leu-Leu-His-Gln-Har-Arg-Lys( )-Trp-Leu-Asn-Asp( )-NH₂ | (SEQ ID NO: 28) |
| H-Ac₅c-Val-Aib-Glu-Ile-Glu-Leu-Leu-His-Gln-Har-Ala-Lys( )-Trp-Leu-Asn-Asp( )-NH₂ | (SEQ ID NO: 29) |
| H-Ac₅c-Val-Aib-Glu-Ile-Gln-Leu-Met-His-Gln-Har-Ala-Lys( )-Trp-Leu-Asp( )-NH₂ | (SEQ ID NO: 30) |
| H-Ac₅c-Val-Aib-Glu-Ile-Gln-Leu-Met-His-Gln-Har-Ala-Lys-Trp-NH₂ | (SEQ ID NO: 31) |
| H-Ser-Val-Ser-Glu-Ile-Gln-Leu-Met-His-Asn-Leu-Gly-Lys-His-Leu-Asn-Ser-Met-Glu- | |
| Arg-Val-Glu-Trp-Leu-Arg-Lys-Lys-Leu-Gln-Asp-Val-His-Asn-Phe-OH | (SEQ ID NO: 32) |
| (PTH(1-34)) | |
| (H-Ac₅c-Val-Aib-Glu-Ile-Gln-Leu-Met-His-Gln-Har-Ala-Lys( )-Trp-Leu-Asp( )-NH₂)₂ | (SEQ ID NO: 33) |
| H-Ac₅c-Val-Aib-Glu-Ile-Gln-Leu-Met-His-Asn-Leu-Gly-Lys( )-His-Leu-Asn-Asp( )-NH₂ | (SEQ ID NO: 34) |
| H-Ser-Val-Ser-Glu-Ile-Gln-Leu-Met-His-Asn-Leu-Gly-Lys( )-His-Leu-Asn-Asp( )-NH₂ | (SEQ ID NO: 36) |
| H-Ser-Val-Ser-Glu-Ile-Gln-Leu-Met-His-Asn-Leu-Gly-Lys-His-Leu-Asn-Ser-NH₂ | (SEQ ID NO: 37) |
| H-Ac₅c-Val-Aib-Glu-Ile-Gln-Leu-Met-His-Gln-Har-Ala-Lys-Trp-Leu-Asn-Asp-OH | (SEQ ID NO: 38) |
| (H-Ac₅c-Val-Aib-Glu-Ile-Gln-Leu-Met-His-Gln-Har-Ala-Lys( )-Trp-Leu-Asn-Asp( )-NH₂)₂ | (SEQ ID NO: 39) |
| H-Ac₅c-Val-Aib-Glu-Ile-Gln-Leu-Met(O)-His-Gln-Har-Ala-Lys( )-Trp-Leu-Asn-Asp( )-NH₂ | (SEQ ID NO: 40) |
| H-Ac₅c-Val-Aib-Glu-Ile-Gln-Leu-Leu-His-Gln-Har-Ala-Dab( )-Trp-Leu-Asn-Asp( )-NH₂ | (SEQ ID NO: 41) |
| H-Ac₅c-Val-Aib-Glu-Ile-Gln-Leu-Met-His-Gln-Har-Ala-Lys-Trp-Leu-Asn-Asp-NH₂ | (SEQ ID NO: 42) |

Where brackets ( ) indicate cyclisation sites.

As described above, the present invention is particularly concerned with PTH analogues in which there is a covalent link between A13 and A17 of the PTH(1-17) analogues. The obtained cyclic conformation of the peptide by said covalent bond has a beneficial effect on the potency of said peptides in vitro, as compared to the similar PTH agonist with no covalent bond. In particular, and more importantly, cyclised analogues have been shown to increase bone mineral density and bone strength in the ovariectomized rat model, an effect which has not been previously shown for linear PTH-analogues shorter than 28 amino acids.

The present inventors also believe that the covalent bond also prevents inhibition of cytochrome P450 2D6 as seen with the linear PTH(1-17) and PTH(1-14) analogues.

As used herein, the term covalent bond may be substituted with the terms cyclisations, links, bonds or bridges without changing the meaning of the word.

There are many possibilities for side-chain-to-side-chain cyclisations including, but not limited to, amides (lactams), esters (lactones), ethers, ketones or disulfides (Synthetic Peptides, A users guide. 2. ed. 2002. Oxford University Press. Ed. Grant, G.A).

The covalent bond between A13 and A17 comprises a lactam bridge or a cysteine bridge.

In a preferred embodiment of the present invention the lactam bond comprises:

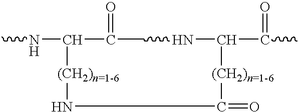

In another aspect of the invention, the lactam bond between Lys13 and Asp17 comprises a process wherein:

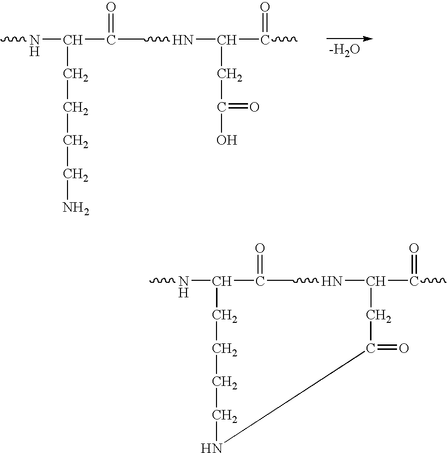

In still another aspect, the present invention relates to the formation of dimers between two PTH analogues.

In one embodiment of the present invention, the dimer formed is a homodimer of the same PTH analogue, such as Ac₅c-Val-Aib-Glu-Ile-Gln-Leu-Met-His-Gln-Har-Ala-Lys-Trp-Leu-Asn-Asp-NH₂(SEQ ID NO: 39), as shown in following scheme:

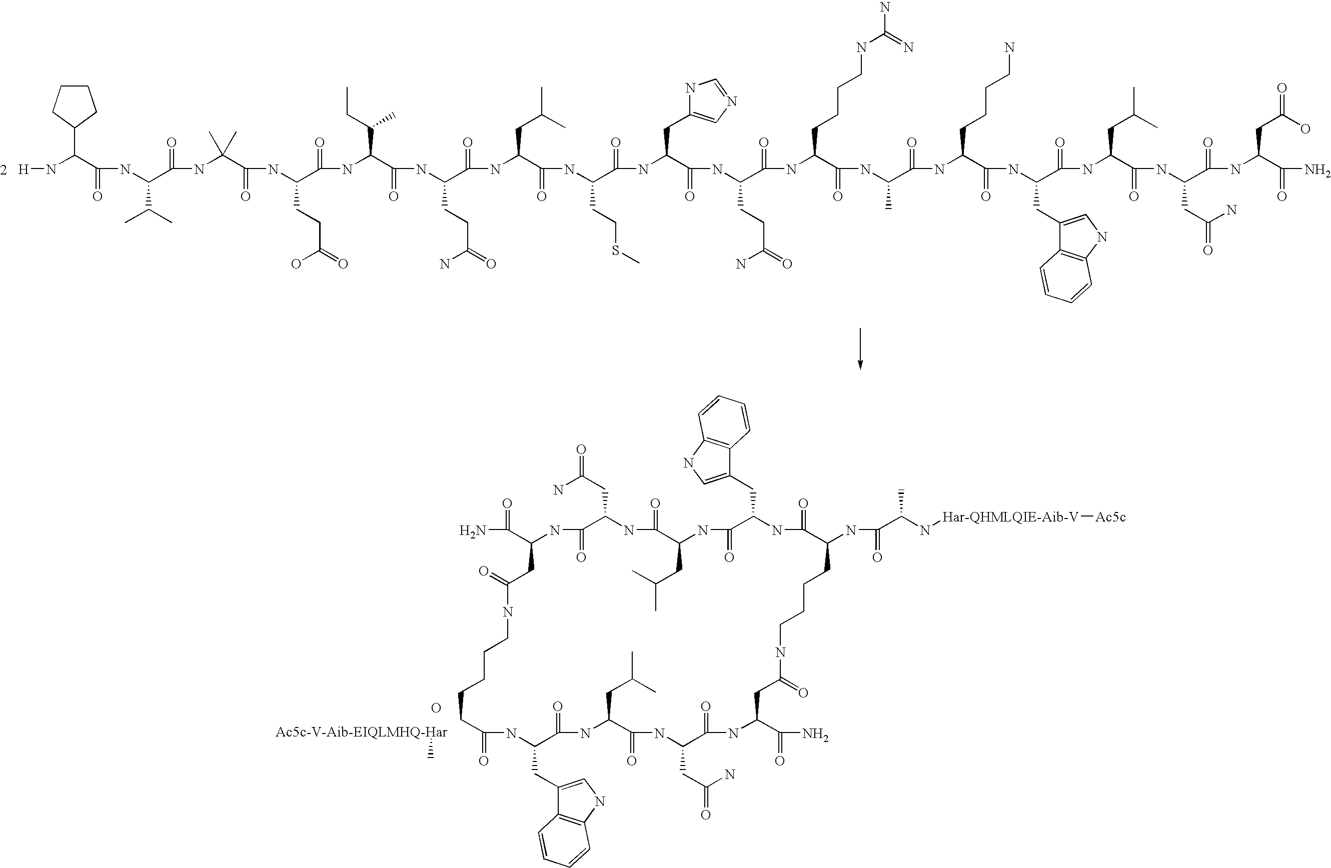

In another embodiment of the present invention, the dimer formed is a heterodimer of two different PTH analogues such as Ac₅c-Val-Aib-Glu-Ile-Gln-Leu-Met-His-Gln-Har-Ala-Lys-Trp-Leu-Asn-Asp-NH₂ and Ac₅c-Val-Aib-Glu-Ile-Gln-Leu-Met-His-Gln-Har-Ala-Lys-Trp-Leu-Asp-NH₂ as shown in the following scheme:

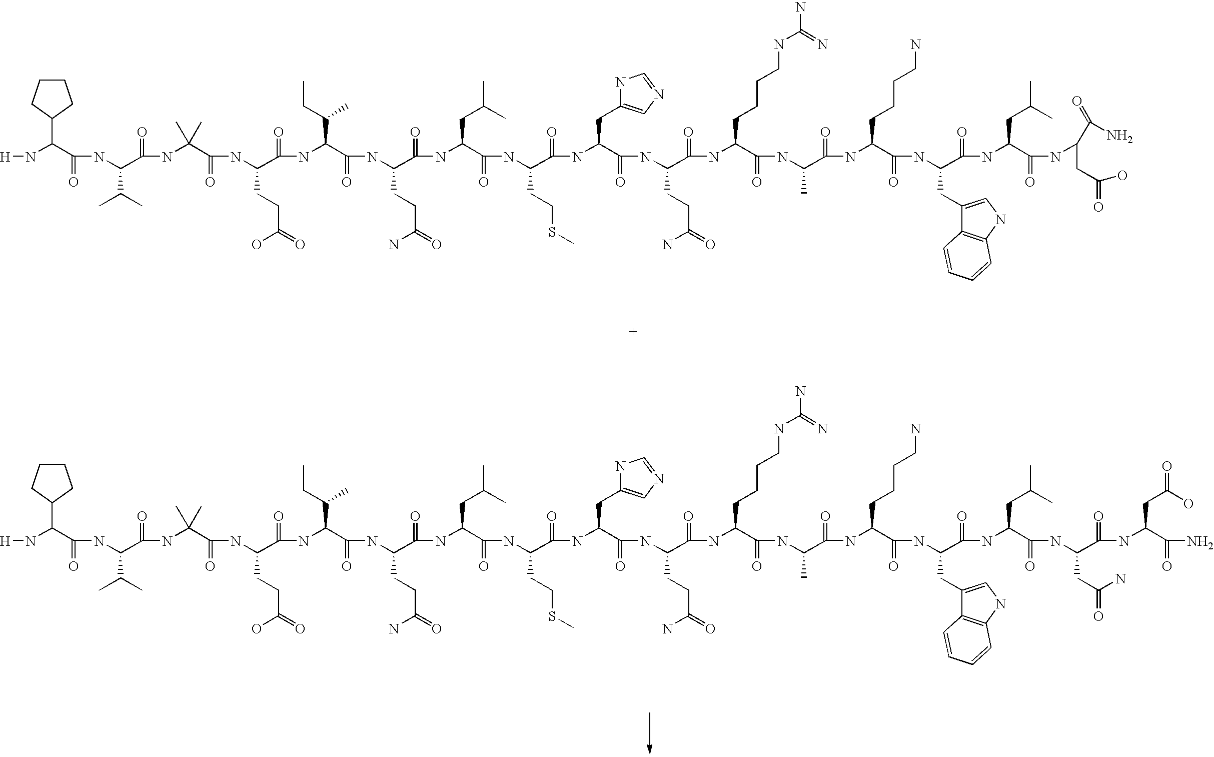

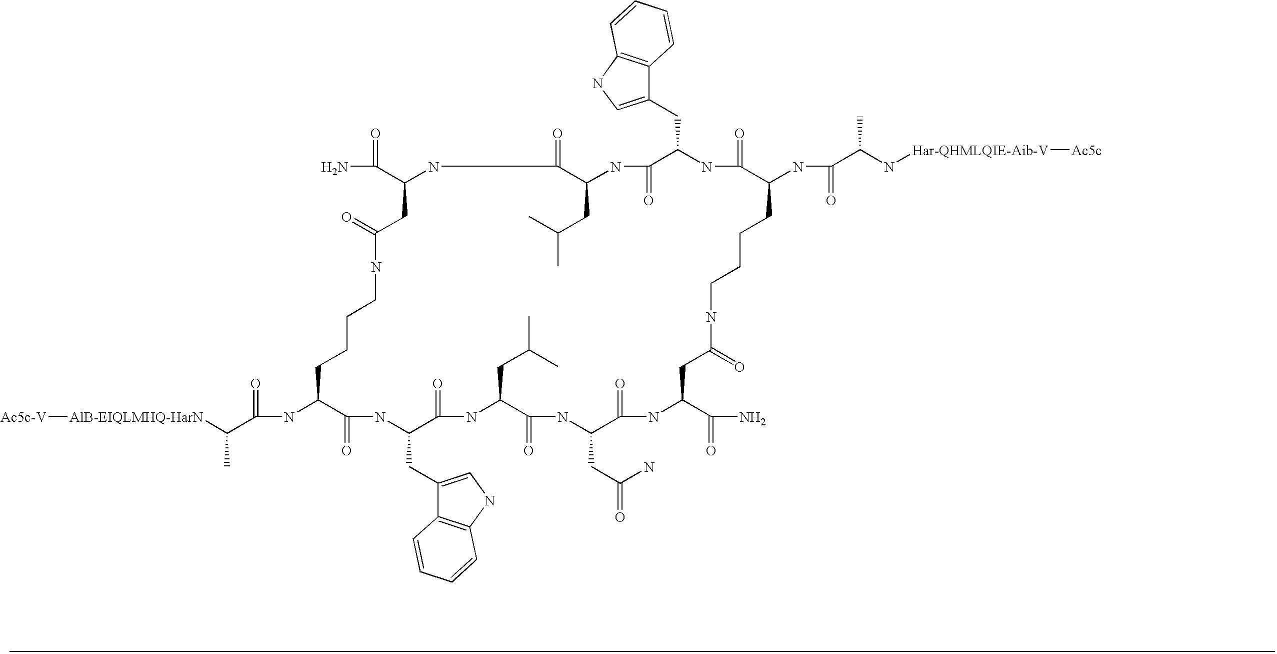

The cyclic conformation of the peptide provided by the covalent bond also prevent against inhibition of cytochrome P450 2D6 as seen with the linear PTH(1-17) analogues. Moreover, a cyclised analogue are shown herein to increase bone mineral density and bone strength in the ovariectomized rat model comparable with PTH(1-34), a result which has not been previously shown for linear PTH(1-17) analogues. Thus, the fact that SEQ ID NO:19 and other semi-cyclic analogues has preserved activity on the PTH receptor and stimulates bone formation in absence of any inhibitory effect on CYP2D6 activity, indicate that prolonged treatment with this compound does not affect the pharmacokinetics of other drugs or herbal products metabolized by this enzyme.

Therefore, we expect that long-term treatment with this novel class of compounds will be associated with increased safety. Furthermore, in the elderly population who are often taken multiple drugs and herbal supplements this feature may be particularly important. Due to little available information on herb-drug interactions, these are often misinterpreted as poor tolerability of the drug. Therefore, the lack of effects of CYP2D6 activity could potentially be important for compliance to the prescribed drug, which in turn may project into better long-term efficacy.

### Medical Indications

The PTH analogues of the present application may be used in but not limited to the prevention or the treatment of conditions such as:

Osteoporosis, such as primary osteoporosis, endocrine osteoporosis (hyperthyroidism, hyperparathyroidism, Cushing's syndrome, acromegaly, type 1 diabetes mellitus, adrenal insufficiency), hereditary and congenital forms of osteoporosis (osteogenesis imperfecta, homocystinuria, Menkes' syndrome, and Riley-Day syndrome), nutritional and gastrointestinal disorders, haematological disorders/malignancy (multiple myeloma, lymphoma and leukaemia, hemophilia, thalassemia), osteoporosis due to immobilization, chronic obstructive pulmonary disease or rheumatologic disorders (rheumatoid arthritis, ankylosing spondylitis).

Osteomyelitis, or an infectious lesion in bone, leading to bone loss.

Hypercalcemia resulting from solid tumours (breast, lung and kidney) and hematologic malignacies (multiple myeloma, lymphoma and leukemia), idiopathic hypercalcemia, and hypercalcemia associated with hyperthyroidism and renal function disorders.

Osteopenia following surgery, induced by steroid administration, and associated with disorders of the small and large intestine and with chronic hepatic and renal diseases.

Osteonecrosis, or bone cell death, associated with traumatic injury or nontraumatic necrosis associated with Gaucher's disease, sickle cell anaemia, systemic lupus erythematosus and other conditions.

Periodontal bone loss.

Osteolytic metastasis.

Bone fracture healing, and

Hyperproliferative skin disorders such as psoriasis.

A preferred indication is osteoporosis, including primary osteoporosis, endocrine osteoporosis (hyperthyroidism, hyperparathryoidism, Cushing's syndrome, and acromegaly), hereditary and congenital forms of osteoporosis (osteogenesis imperfecta, homocystinuria, Menkes' syndrome, and Riley-Day syndrome) and osteoporosis due to immobilization of extremities.

### Pharmaceutical Compositions and Administration:

The PTH analogues of the present invention, or salts or derivatives thereof, may be formulated as pharmaceutical compositions prepared for storage or administration, and which comprise a therapeutically effective amount of a PTH peptide of the present invention, or a salt or derivative thereof, in a pharmaceutically acceptable carrier.

It is within the invention to provide a pharmaceutical composition, wherein the PTH analogue, or a salt thereof is present in an amount effective to regain bone mass in a subject to whom they are administered.

As is apparent to one skilled in the medical art, a “therapeutically effective amount” of the peptides or pharmaceutical compositions of the present invention will vary depending upon the age, weight and mammalian species treated, the particular compounds employed, the particular mode of administration and the desired effects and the therapeutic indication. Because these factors and their relationship to determining this amount are well known in the medical arts, the determination of therapeutically effective dosage levels, the amount necessary to achieve the desired results described herein, will be within the ambit of the skilled person.

As used herein, “a therapeutically effective amount” is one which reduces symptoms of a given condition or pathology, and preferably which normalizes physiological responses in an individual with the condition or pathology. Reduction of symptoms or normalization of physiological responses can be determined using methods routine in the art and may vary with a given condition or pathology.

In one embodiment of the invention administration of the compounds or pharmaceutical composition of the present invention is commenced at lower dosage levels, with dosage levels being increased until the desired physiological effect is achieved. This would define a therapeutically effective amount. For the peptides of the present invention, alone or as part of a pharmaceutical composition, such doses may be between about 0.5 ug/kg/day or 1 ug/kg/day to about 1000.0 ug/kg/day, more preferably, the effective amount of the peptide is about 5.0 ug/kg/day to about 500.0 ug/kg/day, and still more preferably, the effective amount of the peptide is about 10.0 ug/kg/day to about 400.0 ug/kg/day.

For therapeutic use, the chosen PTH analogue is formulated with a carrier that is pharmaceutically acceptable and is appropriate for delivering the peptide by the chosen route of administration. For the purpose of the present invention, the oral, rectal, nasal, or lower respiratory (pulmonary) routes are preferred. These are so-called non-injectable routes. Certain compounds used in the present invention may also be amenable to administration by peripheral parenteral routes include intravenous, intramuscular, subcutaneous, and intra peritoneal routes of administration. The present pharmaceutical composition comprises a PTH analogue of the invention, or a salt or derivative thereof and a pharmaceutically acceptable carrier. Suitable pharmaceutically acceptable carriers are those used conventionally with peptide-based drugs, such as diluents, excipients and the like. Pharmaceutically acceptable carriers for therapeutic use are well known in the pharmaceutical art, and are described, for example, in Remington's Pharmaceutical Sciences, Mack Publishing Company, Easton, Pa., 19th Edition, 1995).

pH buffering agents may be histidine, or sodium acetate. Preservatives, stabilizers, dyes and even flavouring agents may be provided in the pharmaceutical composition. For example, phenol sodium benzoate, sorbic acid and esters of p-hydroxybenzoic acid may be added as preservatives. In addition, antioxidants and suspending agents may be used, e.g. SDS, ascorbic acid, methionine, carboxy methyl cellulose, EDTA, polyethylene glycol, and Tween.

In another embodiment, a pharmaceutically acceptable acid addition salt of the PTH peptide analogue is provided for.

The pharmaceutical compositions of the present invention may be formulated and used as tablets, capsules or elixirs for oral administration; suppositories for rectal administration; sterile solutions and suspensions for injectable administration; inhaleable formulations for nasal or pulmonary administration; and the like.

The dose and method of administration can be tailored to achieve optimal efficacy but will depend on such factors as weight, diet, concurrent medication and other factors, which those skilled in the medical arts will recognize.

When administration route is a non-injectable route, such as oral, rectal, nasal or pulmonary, the pharmaceutical compositions can be prepared in conventional forms. Orally administration may be in liquid formulation or as tablets or capsules. Rectal administration may be as suppositories. Nasal and pulmonary administration can be as liquid or powder.

When administration is to be parenteral, such as subcutaneous on a daily basis, injectable pharmaceutical compositions can be prepared in conventional forms, either as aqueous solutions or suspensions lyophilized, solid forms suitable for reconstitution immediately before use or suspension in liquid prior to injection, or as emulsions. Suitable excipients are, for example, water, saline, dextrose, mannitol, lactose, lecithin, albumin, sodium glutamate, and cysteine hydrochloride. In addition, if desired, the injectable pharmaceutical compositions may contain minor amounts of non-toxic auxiliary substances, such as wetting agents, or pH buffering agents. Absorption enhancing preparations (e.g., liposomes) may be utilized.

In one embodiment of the invention, the compounds are formulated for administration by infusion, e.g., when used as liquid nutritional supplements for patients on total parenteral nutrition therapy, or by injection, for example subcutaneously, intraperitoneal or intravenously, and are accordingly utilized as aqueous solutions in sterile and pyrogen-free form and optionally buffered to physiologically tolerable pH. Formulation for intramuscular administration may be based on solutions or suspensions in plant oil, e.g. canola oil, corn oil or soy bean oil. These oil based formulations may be stabilized by antioxidants e.g. BHA (butylated hydroxianisole) and BHT (butylated hydroxytoluene).

Thus, the present peptide compounds may be administered in a vehicle, such as distilled water or in saline, phosphate buffered saline, 5% dextrose solutions or oils. The solubility of the PTH analogue may be enhanced, if desired, by incorporating a solubility enhancer, such as detergents and emulsifiers.

The aqueous carrier or vehicle can be supplemented for use as injectables with an amount of gelatin that serves to depot the PTH analogue at or near the site of injection, for its slow release to the desired site of action. Alternative gelling agents, such as hyaluronic acid, may also be useful as depot agents.

The PTH analogue may be utilized in the form of a sterile-filled vial or ampoule containing a pharmacologically effective amount of the peptide, in either unit dose or multi-dose amounts. The vial or ampoule may contain the PTH analogue and the desired carrier, as an administration ready formulation. Alternatively, the vial or ampoule may contain the PTH peptide in a form, such as a lyophilized form, suitable for reconstitution in a suitable carrier, such as sterile water or phosphate-buffered saline.

The therapeutic dosing and regimen most appropriate for patient treatment will of course vary with the disease or condition to be treated, and according to the patient's weight and other parameters. Without wishing to be bound by any particular theory, it is expected that doses, in the μg/kg range, and shorter or longer duration or frequency of treatment may produce therapeutically useful results. In some instances, the therapeutic regimen may include the administration of maintenance doses appropriate for preventing tissue regression that occurs following cessation of initial treatment. The dosage sizes and dosing regimen most appropriate for human use may be guided by the results obtained by the present invention, and may be confirmed in properly designed clinical trials.

An effective dosage and treatment protocol may be determined by conventional means, starting with a low dose in laboratory animals and then increasing the dosage while monitoring the effects, and systematically varying the dosage regimen as well. Numerous factors may be taken into consideration by a clinician when determining an optimal dosage for a given subject. Such considerations are known to the skilled person.

### Peptide Synthesis:

The PTH analogues may be synthesized in a number of ways including for example, a method which comprises synthesizing the peptide by means of solid phase or liquid phase peptide synthesis and recovering the synthetic peptide thus obtained. Preferred general procedures are described below. However, more detailed descriptions of solid phase peptide syntheses are found in WO 98/11125.

### Apparatus and Synthetic Strategy

Peptides were synthesized batch wise in a polyethylene vessel equipped with a polypropylene filter for filtration using 9-fluorenylmethyloxycarbonyl (Fmoc) as N-α-amino protecting group and suitable common protection groups for side-chain functionalities.

### Solvents

Acetonitril (HPLC-grade, Sigma-Aldrich, Germany) and NMP (N-methylpyrrolidone, Univar Europe, Denmark) was used directly without purification.

### Amino Acids

Fmoc-protected amino acids were purchased from Advanced ChemTech, Kentucky, USA or Fluka, Germany, in suitable side-chain protected forms. The unnatural amino acids 1-(Fmoc-amino)-cyclopentane-1-carboxylic acid (Ac₅c), Fmoc-aminoisobutyric acid (Aib), Fmoc-Homoarg(pmc)-OH (Har) were together with Fmoc-Lys(Aloc)-OH purchased from Bachem, Germany. Fmoc-Asp(OAll)-OH was obtained from Fluka, Germany.

### Coupling Reagents

Coupling reagent diisopropylcarbodiimide (DIC) was purchased from Fluka, Germany.

### Solid Supports

Peptides were synthesized on TentaGel S Ram resin 0.23 mmol/g (Rapp polymere, Germany).

### Catalysts and Other Reagents

Diisopropylethylamine (DIEA) was purchased from Perspective Biosystem, England, piperidine and pyridine from Riedel-de Haen, Frankfurt, Germany. Ethandithiol was purchased from Aldrich/Fluka, Germany, 1-hydroxybenzotriazole (HOBt) and triisopropylsilane (TIS) from Fluka, Germany. O-(7-azabenzotriazol-1-yl)-1,1,3,3-tetramethyluronium hexafluorophosphate (HATU) was obtained from ChemPep, Miami, USA. N-methylmorpholine was purchased from Lancaster, England. Benzotriazol-1-yl-oxytripyrrolidinophosphonium hexafluorophosphate (PyBop) was obtained from Advanced ChemTech, Kentucky, USA and tetrakis(triphenylphosphine)palladium was obtained from Aldrich, Germany.

### Coupling Procedures

The amino acids were coupled as in situ generated OBt esters made from appropriate N-α-protected amino acids and HOBt by means of DIC in NMP or as in situ generated OAt esters made from appropriate N-α-protected amino acids and HATU by means of DIEA in NMP.

Deprotection of the N-α-amino Protecting Group (Fmoc)

Deprotection of the Fmoc group was performed by treatment with 20% piperidine in NMP (1×5 and 1×10 min.), followed by wash with NMP (5×15 ml, 5 min. each).

### Coupling of OBt-esters

3 eq. N-α-amino protected amino acid was dissolved in NMP together with 3 eq. HOBt and 3 eq DIC and then added to the resin.

### Coupling of OAt-Esters

3 eq. N-α-amino protected amino acid was dissolved in NMP together with 3 eq. HATU and 3 eq DIEA and then added to the resin.

Cleavage of Peptide from Resin with Acid

Peptides were cleaved from the resins by treatment with 92/1/2.5/2.5% v/v trifluoroacetic acid (TFA, Riedel-de Haen, Frankfurt, Germany)/TIS/water/ethandithiol at r.t. for 2 h. The filtered resins were washed with 95% TFA-water and filtrates and washings evaporated under reduced pressure. The residue was precipitated with ether and freeze dried from acetic acid-water. The crude product was analyzed by high-performance liquid chromatography (HPLC) and identified by mass spectrometry (MS).

### Batchwise Peptide Synthesis on TentaGel Resin (PEG-PS)

TentaGel S Ram resin (1 g, 0.23 mmol/g) was placed in a polyethylene vessel equipped with a polypropylene filter for filtration. The resin was swelled in NMP (15 ml), and treated with 20% piperidine in NMP in order to remove the initial Fmoc group on the linker TentaGel S RAM. The resin was drained and washed with NMP. The amino acids according to the sequence were coupled as preformed Fmoc-protected OBt or OAt esters (3 eq.) as described above. The couplings were continued for 2 h, unless otherwise specified. The resin was drained and washed with NMP (5×15 ml, 5 min each) in order to remove excess reagent. Prior to deprotection of the last Fmoc protection group, the lactam bridge was performed on the resin by first deprotection of D(OAll) and K(Aloc) and afterward cyclisation as described below. After completed synthesis, cyclisation and Fmoc deprotection the peptide-resin was washed with NMP (3×15 ml, 5 min each), ethanol (3×15 ml, 1 min each) and finally diethyl ether (3×15 ml, 1 min each) and dried in vacuo. The peptide was cleaved from the resin as described earlier and the crude peptide product was analysed and purified as described below.

### Deprotection of OAll and Aloc

5 eq. of tetrakis(triphenylphosphin)palladium was suspended in a solution comprised of 92.5/5/2.5% v/v chloroform/acetic acid/N-methylmorpholine under a flow of N₂ for 2 min. The suspension was transferred to the peptidylresin placed in a polyethylene vessel plugged in the one end and equipped with a polypropylene filter, and the reaction was allowed to take place under a steam of N₂ 2 h, at r.t. The resin was afterward drained and washed with the above mentioned solution until it turned colourless. Thereafter, the resin was washed with 0.5%, DIEA in NMP (3×5 min.) and finally NMP (3×5 min.).

Cyclisation with Lys and Asp Side Chains

The peptidyl resin with the un-protected D and K was allowed to react with a solution of PyBop, HoBt and DIEA (3 eq. each) in NMP over night. The resin was drained and a fresh portion of the reaction mixture was added for 2 h. Finally the resin was drained and washed with NMP.

### HPLC Conditions

Gradient HPLC analysis was done using a Hewlett Packard HP 1100 HPLC system consisting of a HP 1100 Quaternary Pump, a HP 1100 Autosampler a HP 1100 Column Thermostat and HP 1100 Multiple Wavelength Detector. Hewlett Packard Chemstation for LC software (rev. A.06.01) was used for instrument control and data acquisition. The following columns and HPLC buffer system was used:

Column: LiChrospher 60, 4×250 mm, 10-15 μm

Buffers: A: 0.1% TFA in MQV; B: 0.085% TFA, 10% MQV, 90% MeCN.

Gradient: 0-1.5 min. 0% B
1.5-25 min 0-50% B
25-30 min 50-100% B
30-35 min 100% B
35-40 min 100-0% B
40-45 min 0% B

Flow 1, ml/min, oven temperature 40° C., UV detection: 1=215 nm.

### HPLC Purification of the Crude Peptide

The crude peptide products were purified PerSeptive Biosystems VISION Workstation. VISION 3.0 software was used for instrument control and data acquisition. The following column and HPLC buffer system was used:

Column: VYDAC, C-18, 5×250 mm, 10-15 μm

Buffer system: Buffers: A: 0.1% TFA in MQV; B: 0.085% TFA, 10% MQV, 90% MeCN.

Gradient: 5% B-50% B over 47 min.

Flow: 35 ml/min, UV detection: 1=215 nm and 280 nm.

### Mass Spectroscopy

The peptides were dissolved in super gradient methanol (Labscan, Dublin, Ireland), milli-Q water (Millipore, Bedford, Mass.) and formic acid (Merck, Damstadt, Germany) (50:50:0.1 v/v/v) to give concentrations between 1 and 10 mg/ml. The peptide solutions (20 μl) were analysed in positive polarity mode by ESI-TOF-MS using a LCT mass spectrometer (Micromass, Manchester, UK) accuracy of +/−0.1 μm/z.

### EXAMPLES

### Example 1

### Chemical Synthesis

Peptide synthesis of H-Ac₅c-Val-Aib-Glu-Ile-Gln-Leu-Met-His-Gln-Har-Ala-Lys( )Trp-Leu-Asn-Asp( )-NH₂ (brackets indicates sites of side chain cyclisation) on TentaGel S Ram.

Dry TentaGel S Ram (0.23 mmol/g, 1.2 g) was placed in a polyethylene vessel equipped with a polypropylene filter for filtration and treated as described under “Batehwise peptide synthesis on TentaGel resin” until finishing the coupling of the N-terminal Ac₅c. The three N-terminal amino acids were coupled as OAt-esters using 3 eq of HATU and DIEA (2×2 h), all the other amino acids were coupled as OBt-esters as described above. After coupling the last amino acid the resin was drained, washed with NMP and Allyl and Aloc were deprotected as described under “Deprotection of OAll and Aloc” and the peptide was cyclised as described under “Cyclisation with Lys and Asp side chains”. After cyclisation the last N-terminal Fmoc group was deprotected and the resin was washed with NMP, EtOH and ether and dried in vacuo. The peptide was cleaved from the resin as described above. The crude product was analyzed by HPLC and MS and the purity was found to be 24% and the identity of the peptide was confirmed by MS (found MH⁺ 2071.13, calculated MH⁺ 2071.11). Yield of crude product 259 mg. The peptide was purified to 96% as described above.

### Synthesis of Dimers

Example of synthesizing the homodimer of SEQ ID NO 30 Ac₅c-Val-Aib-Glu-Ile-Gln-Leu-Met-His-Gln-Har-Ala-Lys-Trp-Leu-Asn-Asp-NH₂:

The dimer was synthesized on a TentaGel S Ram resin, and the amino acids are coupled in the following order: D(OAll)-Asn-Leu-Trp-Lys-(Dde-Lys(Fmoc))-(Fmoc-Asp-NH₂)-Asn-Leu-Trp-(Dde-Lys(Fmoc))-Ala-Har-Gln-His-Met-Leu-Gln-Ile-Glu-Aib-Val-Acsc. After coupling of the second Dde-Lys(Fmoc) the cyclisation between Asp and Lys was performed according to the description for the monomer peptide synthesis. Afterwards the Dde protection groups on Lys were removed by agitating the peptidyl resin in 2% hydrazine in NMP (3×5 min.). The following amino acids were then coupled to the free amino group on K in 6 equivalents. Cleavage of the peptide from the resin and the further processing of the homodimer were performed as described for the monomer peptide synthesis.

### Example 2

### In vitro Testing of PTH Analogues in MC3T3-E1 cAMP Efficacy Assay

### Materials and Methods

MC3T3-E1 subclone 4 (mineralizing) (MC3T3-E1) cells (ATCC CRL-2593) were grown in α-MEM (Invitrogen #32571)/10% fetal calf serum+penicillin/streptomycin in a humid atmosphere of 95% air/5% CO₂ at 37° C.

Peptides were dissolved in phosphate buffered saline and further diluted in Tyrode's buffer (TB, Sigma, T2145) containing 0.1% alkali-treated casein (ATC) (Livesey and Donald, Clin. Chim. Acta 1982, 123: 193-8.) and 100 μM isobutyl-methyl-xanthine (IBMX, Sigma 15879).

[¹²⁵I]cAMP FlashPlate® assays (cat. no. 4 SMP100 1A, Perkin Elmer Life Sciences) were used to determine cAMP concentrations.

MC3T3-E1 cells were seeded at 10,000 cells/well in 96-microtiter plates and grown overnight before the efficacy assays. On the day of analysis the growth medium was carefully removed by suction. Cells were washed once with 200 μl TB/0.1% ATC. The buffer was replaced with 100 μl reaction mixture (±test peptide, +100 μM IBMX) and incubated at 37° C. for 13 min. The reaction was stopped by addition of 25 μl of ice-cold 0.5 M HCl. Cells were in the following incubated on ice for 60 min. 75 μl acetate-buffer were added to each well in a 96-well cAMP FlashPlate. 25 μl of the acid cell extract and 100 μl [¹²⁵I] cAMP solution were added onto the same FlashPlate. FlashPlates were incubated overnight at 4° C., emptied by suction and counted in a TopCounter.

### Study Design

Determinations were performed in triplicates at all doses. Standards were included as single determinations, duplicates or triplicates in each experiment, preferentially on each plate.

### Concentration Levels and Groups

Efficacy was evaluated in the concentration range from 10 μM to 10 μM.

### Data Analysis and Statistics

Concentration-dependent cAMP responses were imported into GraphPad Prism vers. 4, transformed and plotted. Curves were fitted with a function for sigmoidal dose response curve non-linear fit Y=Bottom+(Top-Bottom)/(1+10̂((LogEC50-X))) where X is the logarithm of concentration and Y is the response. Y starts at Bottom and goes to Top with a sigmoid shape. pEC50 and the maximally inducible concentration of cAMP were evaluated. Overall statistical differences were analyzed using one-way ANOVA. Post-hoc comparisons were made by the use of Fisher's Least Significance Test. Results were considered significant, when p was lower than 0.05.

### Results

MC3T3-E1 cells were analysed by PCR and shown to express mRNA for the PTH receptor 1 (data not shown). The positive reference compound PTH(1-34) induced a robust cAMP response in this cell line. Several peptides were compared in the cAMP efficacy assay (Table 4).

TABLE 4

Peptides tested in the MC3T3-E1 assay including the
homodimer SEQ ID NO 33.

| | |
|---|---|
| SEQ ID | Sequence |

| | |
|---|---|
| 31 | H-Ac₅c-Val-Aib-Glu-Ile-Gln-Leu-Met-His-Gln-Har-Ala- |
| | Lys-Trp-NH₂ |
| 30 | H-Ac₅c-Val-Aib-Glu-Ile-Gln-Leu-Met-His-Gln-Har-Ala- |
| | Lys( )-Trp-Leu-Asp( )-NH₂ |
| 33 | Dimer H-Ac₅c-Val-Aib-Glu-Ile-Gln-Leu-Met-His-Gln-Har- |
| | Ala-Lys( )-Trp-Leu-Asp( )-NH₂ |
| 2 | H-Ac₅c-Val-Aib-Glu-Ile-Gln-Leu-Met-His-Gln-Har-Ala- |
| | Lys( )-Trp-Leu-Asn-Asp( )-NH₂ |
| 42 | H-Ac₅c-Val-Aib-Glu-Ile-Gln-Leu-Met-His-Gln-Har-Ala- |
| | Lys-Trp-Leu-Asn-Asp-NH₂ |
| 3 | H-Ac₅c-Val-Aib-Glu-Ile-Gln-Leu-Met-His-Gln-Har-Ala- |
| | Lys-Trp-Leu-Asn-Asn-NH₂ |
| 32 | H-Ser-Val-Ser-Glu-Ile-Gln-Leu-Met-His-Asn-Leu-Gly- |
| | Lys-His-Leu-Asn-Ser-Met-Glu-Arg-Val-Glu-Trp-Leu-Arg- |
| | Lys-Lys-Leu-Gln-Asp-Val-His-Asn-Phe-OH |

Brackets indicates cyclisation sites

Potencies and maximal efficacies of cyclic PTH(1-17) analogues and control peptides are shown in Table 5.

TABLE 5
pEC50 and maximum efficacy values ± SEM of novel
PTH analogues in the MC3T3-E1 cAMP efficacy assay.

| | | Emax ± SEM | |
|---|---|---|---|
| SEQ ID | pEC50 ± SEM | [pmol/well] | n |
| 2 | 9.0 ± 0.2 | 4.9 ± 0.8 | 4 |
| 42 | 8.2 ± 0.2 * | 5.7 ± 1.0 | 4 |
| 3 | 8.6 ± 0.1 * | 5.5 ± 0.9 | 4 |
| 32 | 9.1 ± 0.1 | 4.4 ± 0.4 | 4 |
| 31 | 8.4 ± 0.1 * | 4.5 ± 1.0 | 3 |
| 30 | 8.0 ± 0.1 * | 5.0 ± 0.2 | 4 |
| 33 | 8.9 ± 0.1 | 5.0 ± 0.3 | 4 |

Values were compared to PTH(1-34).
*p < 0.05 (Fisher LSD-test); n = number of determinations.

All tested peptides were full agonists on PTH receptor cAMP signalling (Table 5). Their dose response curves could be fitted by a simple sigmoid curve (Hill coefficient close to 1). All data were included in the analysis, i.e. no outliers were removed.

SEQ ID NO: 32 induced cAMP with an EC50 value around 1 nM, consistent with published reports on similar assay systems (Rhee et. al., Yonsei Med. J, 47: 214-22, 2006; Murrills et. al., Bone, 35: 1263-72, 2004.).

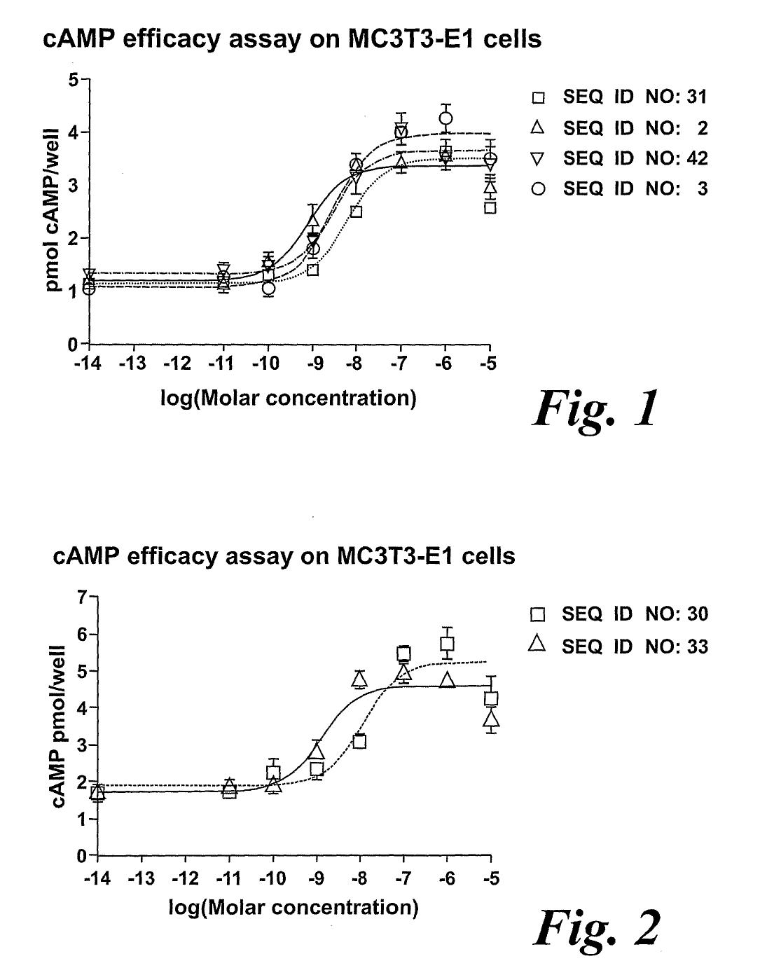

SEQ ID NO: 2 was the most potent cyclic PTH(1-17) analogue among the new, short PTH analogues tested on MC-3T3-E1 cells. SEQ ID NO: 2 had an EC50 comparable to PTH(1-34) and it was significantly more potent than SEQ ID NO: 31, SEQ ID NO: 3 and SEQ ID NO: 42, confirming the importance of cyclisation for agonist activity (FIG. 1; Table 5).

SEQ ID NO: 42 a linear variant of SEQ ID NO: 2 containing the same K13/D17 combination of amino acids able to form a salt-bridge in the C-terminus, was a 6-fold weaker agonist than SEQ ID NO: 2.

The linear analogue SEQ ID NO: 3, a SEQ ID NO: 42 variant without salt-bridge forming capabilities, was 2 to 3-fold weaker than SEQ ID NO: 2.

Remarkably, SEQ ID NO: 33, the covalent dimer of SEQ ID NO: 30 turned into an eight-fold stronger agonist than SEQ ID NO: 30, achieving similar potency to SEQ ID NO: 2 (FIG. 2; Table 5).

### Conclusion

We have identified four novel, short peptide PTH receptor 1 mimetics that exert full agonist responses in an osteoblast-like cell line. Among these the 17-mer cyclised molecules SEQ ID NO: 2 and dimeric SEQ ID NO: 30 were the most potent compounds with an EC50 around-1-nM which is similar to PTH(1-34). K13-D17 cyclisation was associated with increased potency as illustrated by 2-6 fold lower potency of the linear analogues SEQ ID NO: 42 and SEQ ID NO: 3.

### Example 3

### cAMP-Efficacy Assays in Saos-2 Cells

### Study Design

The protocol was adapted from Murrills R. J. et al. (Bone 35: 1263-72, 2004) with modifications. Peptides were dissolved in phosphate buffered saline and further diluted in Tyrode's buffer (TB, Sigma-Aldrich) containing 0.1% BSA and 100 μM isobutyl-methyl-xanthine (IBMX, Sigma-Aldrich). Saos-2 cells were seeded at 50,000 cells/well in 96-microtiter plates and grown for two days before the efficacy assays. On the day of analysis, the growth medium was carefully removed by suction. Cells were washed once with 200 μl TB/0.1% BSA. The buffer was replaced with 100 μl reaction mixture (±test peptide), and incubated at 37° C. for 15 min. The reaction was stopped by addition of 25 μl of ice-cold 0.5 M HCl. Cells were in the following incubated on ice for 60 min. 75 μl acetate-buffer, pH6.2, was added to each well of a 96-well cAMP FlashPlate (Perkin-Elmer). 25 μl of the acid cell extract and 100 μl [¹²⁵I]cAMP solution were added to each well of the same FlashPlate. FlashPlates were incubated overnight at 4° C., emptied by suction and counted in a TopCounter (Packard).

Determinations were performed in single determinations or triplicates at all doses. Z-factor determination indicated that single determinations had a sufficient degree of accuracy to predict potencies of the tested peptides. Standards were included as single determinations, duplicates or triplicates in each experiment, preferentially on each plate.

Efficacy was evaluated at the concentrations 0.01 nM, 0.1 nM, 1 nM, 10 nM, 100 nM, 1 μM and 10 μM, or at 0.1 nM, 1 nM, 3 nM, 10 nM, 30 nM, 100 nM and 1 μM (narrow dose range for simultaneous determination of potencies and Hill-coefficients).

### Data Analysis and Statistics

Concentration-dependent cAMP responses were imported into GraphPad Prism vers. 4 (GraphPad Software), transformed and plotted. Cyclic PTH(1-17) analogues showing potencies greater than 1 μM typically had Hill-coefficients significantly higher than one. Thus, a four-parameter logistic equation was used for determination of the EC50 values. Curves were fitted with a function for sigmoidal dose response curve (variable slope) non-linear fit Y=Bottom+(Top-Bottom)/(1+10̂((LogEC50−X)*HillSlope)) where X is the logarithm of concentration and Y is the response. Y starts at Bottom and goes to Top with a sigmoid shape. pEC₅₀, Hill-coefficient and the maximally inducible concentration of cAMP (Eₘₐₓ) were evaluated.

Overall statistical differences were analyzed with Statistica (Statsoft) using one-way ANOVA. Post-hoc comparisons were made by the use of Fisher's Least Significance Test. Results were considered significant, when p was lower than 0.05.

### Results

### CAMP Efficacy Assay in Saos-2 Cells

A summary of potencies and efficacies of cyclic PTH(1-17) analogues and control peptides is shown in Table 6.

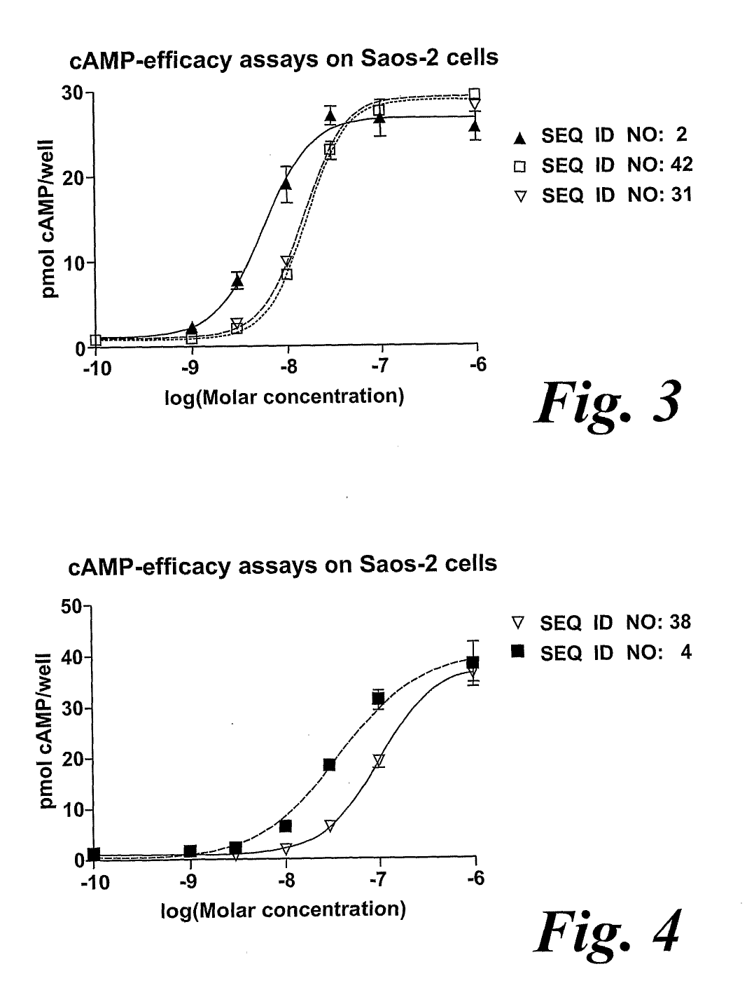

The cyclic, structurally stabilized PTH(1-17) analogue SEQ ID NO: 2 had significantly higher potency in cAMP-efficacy assays in Saos-2 cells than its parent linear PTH(1-17) analogue SEQ ID NO: 42 or the linear PTH(1-14) analogue SEQ ID NO: 31 (FIG. 3).

Similarly, the cyclic, structurally stabilized PTH(1-17) analogue SEQ ID NO: 4 had clearly higher potency in cAMP-efficacy assays in Saos-2 cells than its parent linear PTH(1-17) analogue SEQ ID NO: 38 (FIG. 4).

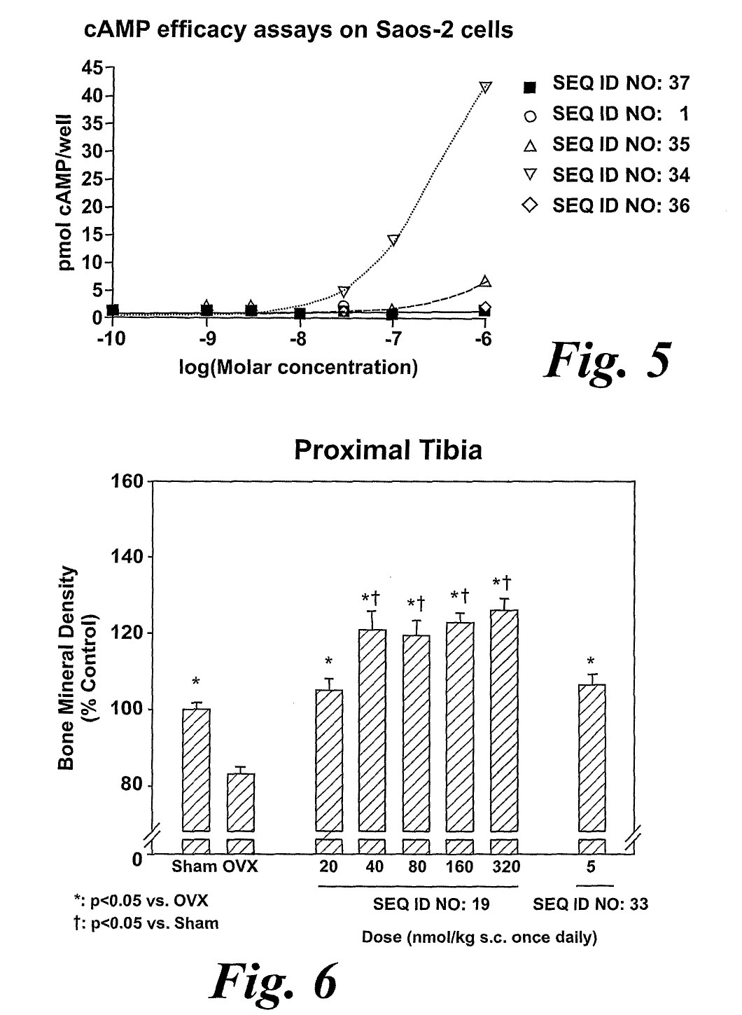

Furthermore, the cyclic, structurally stabilized PTH(1-17) analogue SEQ ID NO: 34 had significantly higher potency in cAMP-efficacy assays in Saos-2 cells than its parent linear PTH(1-17) analogue SEQ ID NO: 1, native, linear PTH(1-17) SEQ ID NO: 37, or analogues of native PTH(1-17) containing either α-helix stabilizing amino acids at positions 1 and 3 (SEQ ID NO: 35), or a covalent bond between side chains of amino acids 13 and 17 (SEQ ID NO: 36), respectively (FIG. 5).

TABLE 6
Potencies and efficacies of relevant
linear and cyclic PTH(1-17) analogues.

| SEQ ID NO | pEC₅₀ ± SEM | Eₘₐₓ ± SEM | N |
|---|---|---|---|
| 1T | inactive | n.a. | 1 |
| 2A | 8.20 ± 0.06 | 0.99 ± 0.03 | 5 |
| 2T | 8.18 ± 0.03 | 0.70 ± 0.09 | 10 |
| 2C | 8.18 ± 0.04 | 0.94 ± 0.03 | 8 |
| 3T | 8.04 ± 0.07 | 1.06 ± 0.06 | 8 |
| 4T | 7.42 ± 0.04 | 1.11 ± 0.03 | 2 |
| 5T | 7.14 ± 0.00 | 1.16 ± 0.12 | 2 |
| 6T | 6.9 | 1.5 | 1 |
| 7T | 7.3 | 1.2 | 1 |
| 8T | 7.94 ± 0.05 | 1.07 ± 0.02 | 2 |
| 9T | 7.85 ± 0.17 | 1.03 ± 0.03 | 2 |
| 10T | 7.90 ± 0.07 | 1.08 ± 0.06 | 3 |
| 11T | 7.17 ± 0.03 | 1.04 ± 0.05 | 2 |
| 12T | 6.99 ± 0.08 | 1.04 ± 0.03 | 2 |
| 13T | 7.27 ± 0.07 | 0.96 ± 0.03 | 3 |
| 14T | 7.45 ± 0.10 | 1.03 ± 0.05 | 3 |
| 15T | 7.22 ± 0.07 | 1.04 ± 0.03 | 3 |
| 16T | 6.92 ± 0.04 | 1.03 ± 0.04 | 2 |
| 17T | 6.11 ± 0.08 | 1.25 ± 0.05 | 2 |
| 18T | 7.29 ± 0.20 | 1.24 ± 0.29 | 2 |
| 19A/T | 7.99 ± 0.03 | 1.05 ± 0.04 | 16 |
| 20T | 8.10 ± 0.05 | 0.97 ± 0.04 | 3 |
| 21T | 6.82 ± 0.34 | 1.40 ± 0.27 | 2 |
| 22T | 7.13 ± 0.05 | 1.08 ± 0.04 | 2 |
| 23T | 7.48 ± 0.07 | 1.16 ± 0.08 | 6 |
| 24T | 6.9 | 1.4 | 1 |
| 25T | 8.05 ± 0.06 | 1.04 ± 0.03 | 8 |
| 26T | 7.90 ± 0.14 | 1.08 ± 0.09 | 8 |
| 27T | 7.91 ± 0.05 | 0.92 ± 0.06 | 3 |
| 28T | 8.27 ± 0.16 | 0.95 ± 0.06 | 3 |
| 29T | 6.95 ± 0.06 | 1.27 ± 0.20 | 2 |
| 30T | 7.09 ± 0.06 | 1.04 ± 0.05 | 3 |
| 31T | 7.66 ± 0.05 | 1.07 ± 0.04 | 8 |
| 32T | 8.61 ± 0.02 | 1.00 ± 0.09 | 21 |
| 33T | 7.88 ± 0.06 | 1.10 ± 0.04 | 9 |
| 34T | 6.67 ± 0.03 | 1.12 ± 0.09 | 2 |
| 35T | 5.09 ± 0.01 | 1.20 ± 0.01 | 2 |
| 36T | inactive | n.a. | 1 |
| 37T | inactive | n.a. | 1 |
| 38T | 7.0 | 1.2 | 1 |
| 39T | 8.30 ± 0.03 | 0.95 ± 0.05 | 6 |
| 40T | 6.9 | 1.1 | 1 |
| 41T | 7.19 ± 0.11 | 0.99 ± 0.02 | 3 |
| 42T | 7.68 ± 0.08 | 1.03 ± 0.07 | 8 |

pEC₅₀: negative logarithm (log) of peptide concentration at half-maximal activation of the receptor; SEM: standard error of the mean; Eₘₐₓ: relative effect to the control (SEQ ID NO: 32); N: number of independent determinations; n.a.: not applicable. Suffixes of SEQ ID NO'S indicate the salt tested (A: acetate; T: trifluoroacetate; C: chloride).

### Conclusions

In three cases (SEQ ID NO's: 2, 4, 34), PTH(1-17)analogues containing α-helix stabilizing, unnatural amino acids as well as cyclisation between side chains of amino acids 13 and 17 showed increased potencies on the Saos-2 PTHLR compared to linear parent analogues, or the native PTH(1-17) peptide containing either α-helix stabilized or a cyclisation without stabilization of the N-terminal α-helix. These results strongly indicate a general positive effect of the amino acid 13 to 17 cyclisation on agonist potency in vitro, when present in conjunction with a stabilized N-terminal α-helix.

Compared to experiments on murine osteoblasts the potency of SEQ ID NO: 32 was reduced by a factor of three. In three specifically tested examples, α-helix stabilized, cyclic analogues of PTH(1-17) (SEQ IDs: 2, 4, 34) showed increased potencies on the Saos-2 PTH1R compared to the native PTH(1-17) peptide and to their linear and/or non α-helix stabilized counterparts. Thus, introduction of a covalent bond between amino acid side chains 13 and 17 further increased the potency of PTH(1-17) analogues.

### Example 4

### In vivo Testing in OVX Rat Model

The ovariectomiced(OVX) rat may be used to test the effect of the PTH analogues on osteopenia/osteoporosis in vivo. The OVX rat develop osteopenia due to ovarian hormone deficiency. Osteopenia can be detected as early as 14 days post OVX, increase for the next 100 days and then stabilized (Wronski T J et al., Calcif. Tissue Int., 43(3): 179-183, 1988). The OVX rat model is considered the “golden standard” by both authorities and industry as a model for osteoporosis (Peter C, Rodan G A. Preclinical safety profile of alendronate. Int j Clin Pract Suppl 1999; 101:3-8, and Stewart A F, Cain R L, Burr D B, Jacob D, Turner C H, Hock J M).

### Study Design

### Animals

One hundred and seventy-eight female Fisher rats were used for the experiment. Initially the animals were housed in Macrolon type 3 cages (2 rats/cage) under controlled conditions (20° C., 55-85% humidity) following a 12:12-hrs light/dark cycle with light on at 6 am. The animals were fed ad libitum with standard Altromin No. 1324 diet (Chr. Petersen, Ringsted, Denmark). The animals had free access to drinking water (domestic quality tap water added citric acid to pH≈3). At the time of inclusion the animals were 6 months old.

### Surgery and Stratification

The week before OVX the animals were stratified according to body weight into two groups that were subjected to either OVX (140 animals) or sham operation (38 animals). Rats were anaesthetized with Hypnorm-Dormicum and the ovariectomy was performed through a midline laparotomy. Sham-operated animals were subjected to midline laparotomy, and the ovaries were exposed but not removed (sham). This group served as an age matched non-osteoporotic control group. A microchip was implanted into each rat during surgery, in order to allow identification of the animals.

To relieve postoperative pain, all rats were treated with buprenorphine (20 mg/100 g s.c. b.i.d.) and meloxicam (0.1 mg/100 g s.c. once daily) for three days after surgery. All rats were allowed 6 days of solitary recovery and then placed in cages two and two.

To allow osteopenia to develop before treatment start, animals were housed for 7 weeks (pre-treatment period) without pharmacological treatment. At the end of the pre-treatment period, sham- and OVX-operated rats were stratified according to bodyweight and divided into six groups of 13-20 animals each (Table 7). At this point, one OVX group (N=20) and one sham group (N=20) were sacrificed. These control groups established baseline levels of bone mineral density (BMD). Furthermore, samples were stored for possible later analysis of bone markers, bone strength, histomorphometry and μCT scans.

For the following 6 weeks one sham group and one OVX group were subjected to vehicle administration (40 mM sodium acetate, 45 mM histidine and 3.9% mannitol, pH 5.5, 300 mOsm/kg). Five OVX groups were treated with increasing doses of the cyclic PTH(1-17) analogue SEQ ID NO: 19. Another OVX group was treated with SEQ ID NO: 33 (Table 7). All drugs were given as s.c. injections. After 6 weeks of treatment the animals were sacrificed. Spine, tibia and femur were collected for analysis of BMD by DEXA-scan and bone samples were stored for later bone strength measurements.

TABLE 7
Dose Levels and Groups

| Groups of Study | | | Dose |
|---|---|---|---|
| (N/group) | Surgery | Substance | (nmol/kg s.c.) |
| Group 1 (N = 17) | SHAM | Vehicle | — |
| Group 2 (N = 18) | OVX | Vehicle | — |
| Group 3 (N = 18) | OVX | SEQ ID NO: 19 | 20 |
| Group 4 (N = 18) | OVX | SEQ ID NO: 19 | 40 |
| Group 5 (N = 18) | OVX | SEQ ID NO: 19 | 80 |
| Group 6 (N = 18) | OVX | SEQ ID NO: 19 | 160 |
| Group 7 (N = 18) | OVX | SEQ ID NO: 19 | 320 |
| Group 8 (N = 13) | OVX | SEQ ID NO: 33 | 5 |
| Group 9 (N = 20) | OVX | Sacrificed when | — |
| Group 10 (N = 20) | SHAM | dosing started | — |

### Data Collection, in Life Period

During the pre-treatment period the animal body weight was recorded twice weekly. The GEDACO data collection system was used for all in vivo data collection in the pre-treatment period. During the dosing period body weight was recorded daily. The GEDACO data collection system was used for all in vivo data collection during the treatment period. A log was kept every day describing any adverse event not recorded in the database.

### Vivisection

On day 10 before sacrifice, all animals were subjected to administration of tetracycline (20 mg/kg i.p.) and on day 2 to administration of calcein (15 mg/kg i.p.). During the week before initiation of compound treatment one group of OVX animals and one group of SHAM animals were sacrificed (groups 9-10). At the end of the dosing period, the remaining animals were sacrificed.

Lumbar vertebrae (L4-L5-L6-S1), left femur and tibia were collected, cleaned, packed in saline moistened gaze in a tube and stored at −20° C. for later ex vivo bone strength measurements. Right femur and tibia as well as lumbar vertebrae (T13-L1-L2-L3) and caudal vertebrae (S2-S3-C1-C2-C3) were collected, cleaned and stored in 70% ethanol for analysis of bone mineral density (BMD) and possible later histomorphometry and/or μCT scanning.

### Determination of Bone Mineral Density by DEXA

Ex vivo BMD measurements were performed at the end of the study using a Lunar Piximus II densitometer (GE Healthcare, Chalfont St. Giles, UK) with a precision of 1.5%. Calibration of the instrument was performed with an aluminium/Lucite phantom.

Tibiae, femora and lumbar (L1-L2) and sacral/caudal (S3-C1) spine fragments were placed on the imaging positioning tray and scanned four times. All specimens were placed in a similar orientation for correct comparison. Regions of interest (ROI) were generated on the scans using the Piximus image analysis software provided with the instrument. The ROIs were defined as proximal tibia below the growth plate (2 mm section), femoral head, femoral shaft (mid third) and two vertebral bodies of the lumbar spine (excluding the dorsal spines).

### Measurement of Bone Strength

Bone strength measurements were performed on a compression device (Lloyd Instruments, Fareham, UK). Bones were positioned with the help of custom-made holders, in order to achieve maximal reproducibility. Maximal force to fracture was determined.

### Data Analysis and Statistics

Overall comparison among groups was performed using one-way ANOVA for one-way classified data (BMD). For individual comparisons among groups, post-hoc analysis was performed using Fisher's least significant difference test. Differences were considered significant at the 5% level. All data are presented as mean±SEM.

### Results

### OVX Study

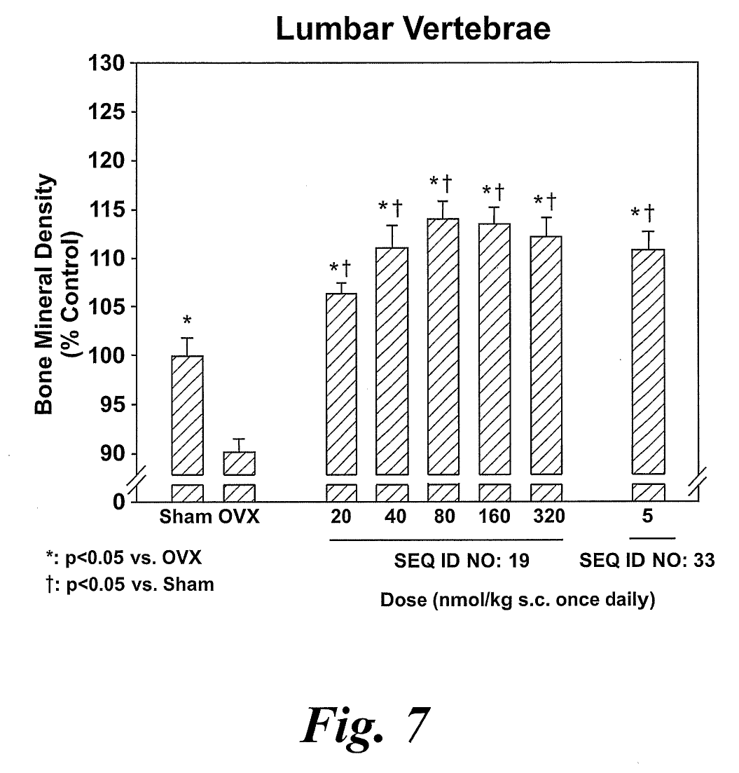

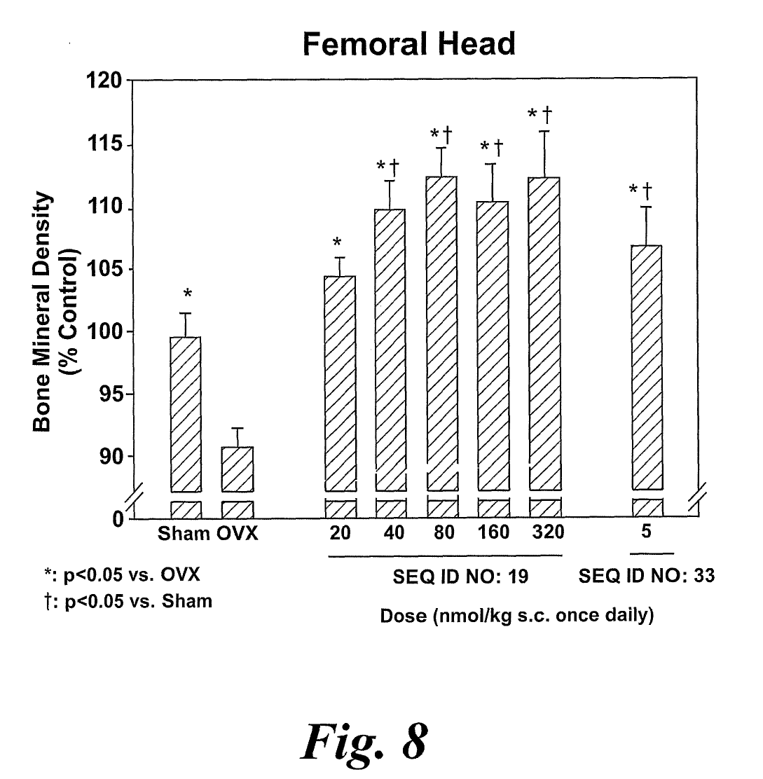

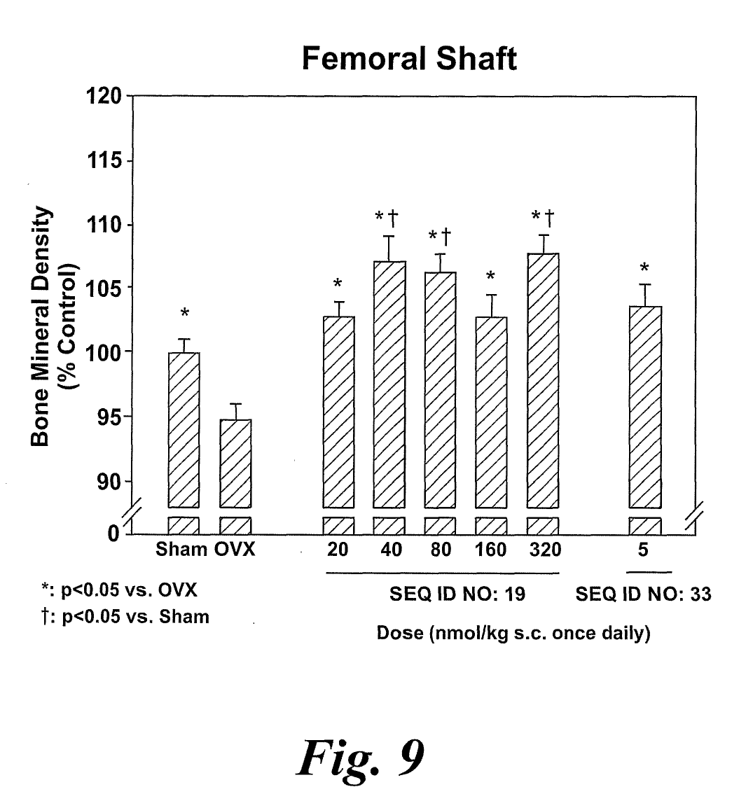

Bone mineral density was evaluated at various sites representing cortical (femoral shaft) and predominantly trabecular (proximal tibia, femoral head, lumbar vertebrae) bone. Regardless of the site investigated, experimental groups treated with the α-helix stabilized, cyclic PTH(1-17) analogue SEQ ID NO: 19 gained significantly higher bone mineral density than vehicle-treated ovariectomized rats at doses of 20 nmol/kg/d to 320 nmol/kg/d (FIGS. 6-9). At doses greater than 20 nmol/kg/d the bone-anabolic effect of SEQ ID NO: 19 even led to bone mineral densities higher than in vehicle-treated sham-operated rats (FIGS. 6-9).

The homodimer SEQ ID NO: 33 also led to significant increases in bone mineral density over vehicle-treated ovariectomized rats at doses of 5 nmol/kg/d at all sited tested (FIGS. 6-9).

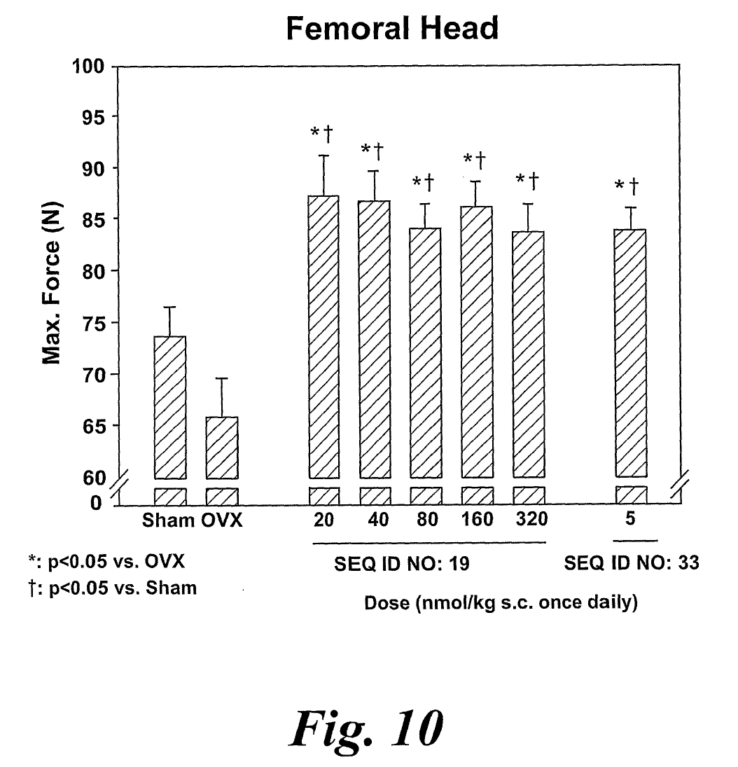

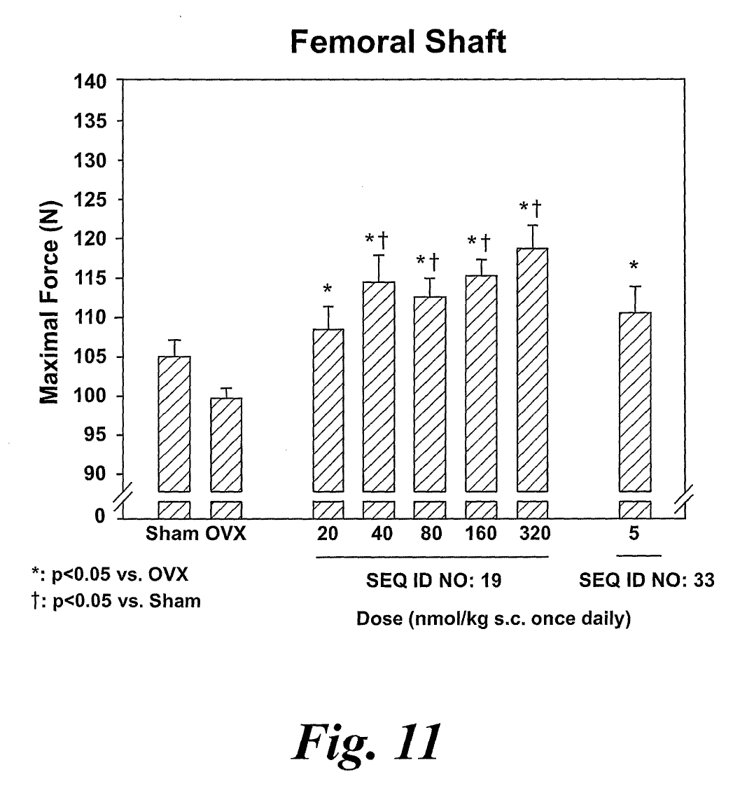

Furthermore, bone strength of the femur in the shaft region (cortical bone) and the femoral head region (trabecular bone) was significantly increased over the level observed in the vehicle-treated OVX-group in the groups treated with SEQ ID NO: 19 and SEQ ID NO: 33 (FIGS. 10 and 11). In the groups treated with SEQ ID NO: 19 at doses greater than 20 nmol/kg/d bone strength was significantly increased over the level of the vehicle-treated sham-operated group (FIGS. 10 and 11).

### Conclusions

One PTH(1-17)analogue containing α-helix stabilizing, unnatural amino acids as well as cyclisation between side chains of amino acids 13 and 17 (SEQ ID NO: 19) showed bone-anabolic activity in cortical and trabecular bone. The anabolic activity led to at least normalization of bone mineral densities at the lowest dose (20 nmol/kg/d), but could be increased by higher doses to values significantly higher than observed in control animals. Thus, SEQ ID NO: 19 is the shortest PTH-analogue with proven anabolic activity to date. In contrast, bone-anabolic activity was absent from a PTH(1-14) analogue containing unnatural α-helix-stabilizing amino acids and without the ability to form the cyclic structure, albeit this PTH-analogue activated the cAMP pathway of the PTH1R receptor with similar potency as our PTH(1-17) analogue (SEQ ID NO 19) (Rhee, Y. et al., (2006) Yonsei Medical Journal, 47, 214-222).

A dimeric PTH(1-17) analogue (SEQ ID NO: 33) was also efficient, and significantly increased bone mineral density and bone strength compared to vehicle-treated OVX-group at a dose of 5 nmol/kg/d. Thus, our design of short PTH analogues has led to a novel class of bone-anabolic PTH analogues that may be useful for the treatment of diseases associated with bone loss, such as postmenopausal osteoporosis.

### Example 5

### In vitro Testing of CYP2D6 Inhibition

### Method and Materials

All chemicals and reagents used for the CYP2D6 inhibition assay are presented in Table 8. Stock solutions of the test compounds (1 mM) were prepared in 50% isopropanol or 20% DMSO. Pooled human liver microsomes (HLM, final concentration 0.05 mg protein/mL) were mixed with phosphate buffer (0.1 M potassium phosphate, pH 7.4), CYP2D6 substrate (dextrometrorphan, 5 μM final concentration) and test compound (10 μM), quinidine (0.5 μM) or vehicle (0.2% DMSO or 0.5% isopropanol). The mixture was preincubated for 5 min at 37° C. prior to initiation of the reaction addition of a NADPH regenerating mixture (Final concentration: 1.25 mM NADP⁺, 3.3 mM Glucose-6-phosphate, 3.3 mM MgCL₂ and 0.4 U/mL glucose-6-phosphat dehydrogenase). After 5 minutes the reaction was stopped by addition of 0.25 volume stop reagent (94% acetonitrile, 6% acetic acid). Each experiment was performed in duplicate or triplicate.

The quantification of CYP2D6 product (dextrorphan) was performed by LC/MS/MS. After centrifugation for 5 min at 10.000 g, 40 μL of the supernatant was injected onto a C8 RP-HPLC column (XterraMS, C8, 2.5 μM, 50×2.1 mm). Dextrorphan was eluted by a linear gradient from 0 to 90% acetonitrile in 0.1% formic acid over 4 min using a flow rate of 0.15 ml/min. The concentration of dextrorphan in the reaction mixture was estimated from the peak area of the MS/MS transition (m/z 258.1>199) using an external calibration curve (Table 8).

TABLE 8
The chemicals and reagents used for the CYP2D6 inhibition assay

| Chemical/reagent | Product # | Supplier |
|---|---|---|
| Acetonitrile | A3485 | Sigma-Aldrich |
| Dextromethorphan hydrobromide | D1053 | |
| monohydrate | | |
| Dextrorphan tartrate | D0127 | |
| Quinidine | Q3625 | |
| Potassium phosphate, monobasic | 0240 | J. T. Baker |
| NADPH Regenerating System A | 451220 | BD Biosciences |
| NADPH Regenerating System B | 451200 | |
| Pooled human liver microsomes | 452161 | |
| Methanol | C26C11X | LAB-SCAN |
| Isopropanol | C19C11X | |
| DMSO | 41650 | Fluca |
| Water purified to a resistance >18MΩ and total | | Millipore |
| organic carbon <7 ppb | | |
| Acetic acid 100% | 1.00056 | Merck |
| Formic acid 98-100% | 1.00264 | |

### Data Analysis

The CYP2D6 activity in HLM was calculated as the metabolite formation rate:

$$v ( pmol \text{/} min \text{/} mg ) = \frac{[ metabolite ]}{t \times [ protein ]}$$

Where, v is the formation rate, [metabolite] is the detected concentration of dextrorphan (pmol/mL) at the time (t, min) and [protein] is the protein concentration (mg/mL) in the reaction mixture. The influence of the test compounds on the CYP2D6 activity was calculated as % of the activity measured when incubated with the vehicle. The effect of CYP2D6 inhibitor (quinidine) was included as inhibition control within each batch analysis. A reduction in CYP2D6 activity to less than 70% of the vehicle control was considered significant.

### Results

Linear truncated PTH compounds reduced the CYP2D6 activity to 22-57% relative to the activity observed when incubated with vehicle (SEQ ID NO 31, 42, 3, 37, 1, and 35, Table 9). When an intra-molecular cyclisation was introduced between amino acid position 13 and 17, the inhibitory effects on CYP2D6 was markedly reduced. i.e. SEQ ID NO:42 (linear, 29%) vs. SEQ ID NO: 2 (cyclic, 67%) and SEQ ID NO: 1 (linear, 57%) vs. SEQ ID NO: 34 (cyclic, 107%). The beneficial effect was not linked to cyclisation between specific amino acids as cyclisation between Lys→Asp, Cys→Cys, Glu→Lys or Lys→Glu were all found to improve CYP2D6 activity (Table 9).

TABLE 9

Effect on intramolecular cyclisation on the CYP2D6
mediated formation rate of dextrorphan upon incubation
of various compounds in a concentration of 10 μM. ( )
denotes intramolecular side-chain cyclisation.

| | | |
|---|---|---|
| | | CYP2D6 |
| SEQ ID | | activity (% |
| NO | Sequence | of vehicle) |

| | | |
|---|---|---|
| 31 | H-AC5C-V-Aib-EIQLMHQ-Har-AKW-NH2 | 22 |
| 42 | H-Ac5c-V-Aib-EIQLMHQ-Har-AKWLND-NH2 | 29 |
| 3 | H-Ac5c-V-Aib-EIQLMHQ-Har-AKWLNN-NH2 | 21 |
| 30 | H-AC5C-V-Aib-EIQLMHQ-Har-AK( )WLD( )-NH2 | 83 |
| 2 | H-AC5C-V-Aib-EIQLMHQ-Har-AK( )WLND( )-NH2 | 67 |
| 7 | H-AC5C-V-Aib-EIQLMHQ-Har-AC( )WLNC( )-NH2 | 85 |
| 8 | H-AC5C-V-Aib-EIQLMHQ-Har-AE( )WLNK( )-NH2 | 99 |
| 9 | H-AC5C-V-Aib-EIQLMHQ-Har-AK( )WLNE( )-NH2 | 85 |
| 37 | H-SVSEIQLMHNLGKHLNS-NH2 | 41 |
| 1 | H-Ac5c-V-Aib-EIQLMHNLGKHLND-NH2 | 57 |
| 35 | H-Ac5c-V-Aib-EIQLMHNLGKHLNS-NH2 | 48 |
| 34 | H-Ac5c-V-Aib-EIQLMHNLGK( )HLND( )-NH2 | 107 |
| 36 | H-SVSEIQLMHNLGK( )HLND( )-NH2 | 79 |

Synergistic effects were found when the intra-molecular cyclisation was combined by specific amino acid substitutions. The substitution of Gln⁶ with Glu, Met⁸ with Leu, Nle or Val, Gln¹⁰ with Glu or C-terminal de-amidation totally eliminated the inhibition of the CYP2D6 (Table 10).

TABLE 10

Effect on single amino acid substitutions on the the CYP2D6
mediated formation rate of dextrorphan upon incubation of
various compounds in a concentration of 10 μM. ( ) denotes
intramolecular side-chain cyclisation.

| | | |
|---|---|---|
| | | CYP2D6 |
| SEQ ID | | activity (% |
| NO | Sequence | of vehicle) |

| | | |
|---|---|---|
| 2 | H-AC5C-V-Aib-EIQLMHQ-Har-AK( )WLND( )-NH2 | 67 |
| 4 | H-AC5C-V-Aib-EIQLMHQ-Har-AK( )WLND( )-OH | 89 |
| 18 | H-Ac5c-V-Aib-EIELMHQ-Har-AK( )WLND( )-NH2 | 89 |
| 19 | H-Ac5c-V-Aib-EIQLLHQ-Har-AK( )WLND( )-NH2 | 90 |
| 20 | H-Ac5c-V-Aib-EIQL-Nle-HQ-Har-AK( )WLND( )-NH2 | 99 |
| 21 | H-Ac5c-V-Aib-EIQLVHQ-Har-AK( )WLND( )-NH2 | 92 |
| 24 | H-Ac5c-V-Aib-EIQLMHE-Har-AK( )WLND( )-NH2 | 83 |
| 41 | H-Ac5c-V-Aib-EIQLLHQ-Har-A-Dab( )-WLND( )-NH2 | 82 |
| 17 | H-Ac5c-V-Aib-EIQLLHQ-Har-A-Dpr( )-WLND( )-NH2 | 101 |

### Conclusions

The linear truncated PHT analogues were found to inhibit CYP2D6, when an intramolecular cyclisation was introduced between amino acid position 13 and 17, the inhibition was markedly reduced. The inhibition could be further reduced by specific amino acid substitutions in position 6, 8, 10 and C-terminal modification.

While the invention has been described in conjunction with the exemplary embodiments described above, many equivalent modifications and variations will be apparent to those skilled in the art when given this disclosure. Accordingly, the exemplary embodiments of the invention set forth are considered to be illustrative and not limiting. All documents cited herein are expressly incorporated by reference.

1. A biologically active PTH(1-17)analogue peptide represented by Formula I which consists of:
R1-Z1-A1-A2-A3-A4-A5-A6-A7-A8-A9-A10-A11-A12-A13-A14-Leu-A16-A17-Z2-R2
wherein
R1 is hydrogen, NH₂, RHN, RR3N, wherein each of R and R3 independently represent C1-4 alkyl (e.g. methyl), acetyl, formyl, benzoyl or trifluoroacetyl;
A1 is Ac5c, Gly, Ser, Ala or any alpha-helix stabilizing residue;
A2 is Val or a conservative substitution;
A3 is Aib, Ala, Ser or any alpha-helix stabilizing residue;
A4 is Gln, Glu or a conservative substitution;
A5 is Ile or a conservative substitution;
A6 is Gln, Glu or a conservative substitution;
A7 is Leu or Phe or a conservative substitution;
A8 is Met, Leu, Nle, Val or a conservative substitution;
A9 is His or a conservative substitution;
A10 is Gln, Glu, Asp, Ala, Val or a conservative substitution;
A11 is Har, Arg, Ala, Ile, Lys or a conservative substitution;
A12 is Ala, Arg, His or a conservative substitution;
A13 is Lys, Orn, Asp, Glu, Cys, Dab or Dpr;
A14 is Trp, Phe, Leu, Arg, His or a conservative substitution;
A16 is Asn, Asp, a conservative substitution or absent;
A17 is, Lys, Orn, Glu, Cys, Asp, Dab or Dpr; and
R2 is OH, OR, NRH, NRR3 or NH₂, wherein each of R and R3 independently represents C1-4 alkyl (e.g. methyl); and
A13 and A17 are linked by one or more covalent bonds; and
Z1 and Z2 are independently absent, or a peptide sequence of 1-10 amino acid units selected from the group consisting of Ala, Leu, Met, Gln, Glu, Lys, Dab, Dpr and Orn;
or a homodimer, heterodimer, or pharmaceutically acceptable salt or derivative thereof.

2. A biologically active PTH(1-17) analogue peptide represented by Formula II which consists of:
R1-Z1-A1-Val-A3-Glu-Ile-A6-A7-A8-His-A10-A11-A12-A13-A14-Leu-A16-A17-Z2-R2
wherein
R1 is hydrogen, NH₂, RHN, RR3N, wherein each of R and R3 independently represent C1-4 alkyl (e.g. methyl), acetyl, formyl, benzoyl or trifluoroacetyl;
A1 is Ac5c, Gly, Ser, Ala or any alpha-helix stabilizing residue;
A3 is Aib, Ala, Ser or any alpha-helix stabilizing residue;
A6 is Gln or Glu;
A7 is Leu or Phe;
A8 is Met, Leu, Nle or Val;
A10 is Gln, Glu, Asp, Ala or Val;
A11 is Har, Arg, Ala, Ile or Lys;
A12 is Ala, Arg or His;
A13 is Lys, Orn, Asp, Glu, Cys, Dab or Dpr;
A14 is Trp, Phe, Leu, Arg or His;
A16 is Asn, Asp or absent;
A17 is Lys, Orn, Glu, Cys, Asp, Dab or Dpr;
R2 is OH, OR, NRH, NRR3 or NH₂, wherein each of R and R3 independently represents C1-4 alkyl (e.g. methyl); and
A13 and A17 are linked by one or more covalent bonds; and
Z1 and Z2 are independently absent, or a peptide sequence of 1-10 amino acid units selected from the group consisting of Ala, Leu, Lys, Dab, Dpr and Orn;
or a homodimer, heterodimer, or pharmaceutically acceptable salt or derivative thereof.

3. A biologically active PTH(1-17) analogue peptide represented by Formula III which consists of:
R1-Z1-Ac5c-Val-Aib-Glu-Ile-A6-Leu-A8-His-A10-A11-Ala-A13-A14-Leu-A16-A17-Z2-R2
wherein
R1 is hydrogen, NH₂, RHN, RR3N, wherein each of R and R3 independently represent C1-4 alkyl (e.g. methyl), acetyl, formyl, benzoyl or trifluoroacetyl;
A6 is Glu or Gln;
A8 is Met, Leu, Nle or Val;
A10 is Gln or Glu;
A11 is Har or Arg;
A13 is Lys, Orn, Asp, Glu, Cys, Dab or Dpr;
A14 is Trp or Phe;
A16 is Asn, Asp, or absent;
A17 is Lys, Orn, Glu, Cys, Asp, Dab or Dpr;
R2 is OH, OR, NRH, NRR3 or NH₂, wherein each of R and R3 independently represents C1-4 alkyl (e.g. methyl); and
A13 and A17 are linked by a covalent bond; and
Z1 and Z2 are independently absent, or a peptide sequence of 1-10 amino acid units selected from the group consisting of Ala, Leu, Lys, Dab, Dpr and Orn;
or a homodimer, heterodimer or pharmaceutically acceptable salt or derivative thereof.

4. A biologically active PTH(1-17) analogue peptide represented by Formula IV which consists of:
R1-Z1-A1-Val-A3-Glu-Ile-A6-A7-A8-His-A10-A11-A12-A13-A14-Leu-A16-A17-Z2-R2
wherein
R1 is hydrogen, NH₂, RHN, RR3N, wherein each of R and R3 independently represent C1-4 alkyl (e.g. methyl), acetyl, formyl, benzoyl or trifluoroacetyl;
A1 is Ac5c, Ac6c, Abu, Nva, Aib;
A3 is Ac5c, Aib, Abu, Nva;
A6 is Gln or Glu
A7 is Leu or Phe;
A8 is Met, Leu, Val or Nle;
A10 is Gln or Glu
A11 is Har or Arg;
A12 is Ala or Arg;
A13 is Lys, Glu, Asp or Cys;
A14 is Trp or Phe,
A16 is Asn, Asp or absent;
A17 is, Glu, Cys, Asp or Lys;
R2 is OH, OR, NRH, NRR3 or NH₂, wherein each of R and R3 independently represents C1-4 alkyl (e.g. methyl); and
A13 and A17 are linked by one or more covalent bonds; and
Z1 and Z2 are independently absent, or a peptide sequence of 1-10 amino acid units selected from the group consisting of Ala, Leu, Lys, Dab, Dpr and Orn;
or a homodimer, heterodimer, or pharmaceutically acceptable salt or derivative thereof.

5. A biologically active PTH(1-17) analogue peptide represented by Formula V which consists of:
R1-Z1-A1-Val-Aib-Glu-Ile Gln-A7-A8-His-Gln-A11-A12-A13-Trp-Leu-A16-A17-Z2-R2
wherein
R1 is hydrogen, NH₂, RHN, RR3N, wherein each of R and R3 independently represent C1-4 alkyl (e.g. methyl), acetyl, formyl, benzoyl or trifluoroacetyl;
A¹ is Ac5c or Ac6c;
A7 is Leu or Phe;
A8 is Met, Leu or Nle;
A11 is Har or Arg;
A12 is Ala or Arg;
A13 is Lys or Glu;
A16 is Asn or absent;
A17 is, Glu, or Asp;
R2 is OH, OR, NRH, NRR3 or NH₂, wherein each of R and R3 independently represents C1-4 alkyl (e.g. methyl); and
A13 and A17 are linked by one or more covalent bonds; and
Z1 and Z2 are independently absent, or a peptide sequence of 1-10 amino acid units selected from the group consisting of Ala, Leu, Lys, Dab, Dpr and Orn;
or a homodimer, heterodimer, or pharmaceutically acceptable salt or derivative thereof.

6. The PTH(1-17) analogue peptide according to any one of claims 1 to 5, wherein the one or more covalent bonds between the amino acid residues at position 13 and position 17 comprises a lactam bridge or a cysteine bridge.

7. The PTH(1-17) analogue peptide according to claim 6, wherein the covalent bond between position 13 and position 17 is a lactam bridge.

8. The PTH(1-17) analogue peptide according to claim 6, wherein the one or more covalent bonds are formed between two PTH analogues.

9. The PTH(1-17) analogue peptide according to any one of the preceding claims which comprises between two and 14 substitutions relative to wild type human PTH(1-17) between residues A1 and A17 inclusive.

10. The PTH(1-17) analogue peptide according to any one of the preceding claims which comprises 1 or 2 substitutions at positions 1 or 3 relative to wild-type PTH, optionally in combination with 1, 2, 3, 4, 5, 6, 7, 8, 9 or 10 substitutions at further positions, including position 6, 7, 8, 10, 11, 12, 13, 14, 16 or 17.

11. The PTH(1-17) analogue peptide according to any one of the preceding claims which comprises substitutions at positions 1 to 10 relative to wild type PTH, optionally in combination with substitutions at one or more further positions, including 11, 12, 13, 14, 16 or 17.

12. The PTH(1-17) analogue peptide according to any one of the preceding claims which comprises substitutions at position 13 with Asp, Lys, Orn, Glu, or Cys and/or substitution of position 17 with Lys, Asp, Orn, Glu or Cys.

13. The PTH(1-17) analogue peptide according to any one of claims 1 to 12 comprising one of the following combinations of residues:

| | |
|---|---|
| H-Ac₅c-Val-Aib-Glu-Ile-Gln-Leu-Met-His-Gln-Har-Ala-Lys( )Trp-Leu-Asn-Asp( )-NH₂ | (SEQ ID NO: 2) |
| H-Ac₅c-Val-Aib-Glu-Ile-Gln-Leu-Met-His-Gln-Har-Ala-Glu( )Trp-Leu-Asn-Lys( )-NH₂ | (SEQ ID NO: 8) |
| H-Ac₅c-Val-Aib-Glu-Ile-Gln-Leu-Met-His-Gln-Har-Ala-Lys( )Trp-Leu-Asn-Glu( )-NH₂ | (SEQ ID NO: 9) |
| H-Ac₆c-Val-Aib-Glu-Ile-Gln-Leu-Leu-His-Gln-Har-Ala-Lys( )Trp-Leu-Asn-Asp( )-NH₂ | (SEQ ID NO: 10) |
| H-Ac₅c-Val-Aib-Glu-Ile-Gln-Leu-Leu-His-Gln-Har-Ala-Lys( )Trp-Leu-Asn-Asp( )-NH₂ | (SEQ ID NO: 19) |
| H-Ac₅c-Val-Aib-Glu-Ile-Gln-Leu-Nle-His-Gln-Har-Ala-Lys( )Trp-Leu-Asn-Asp( )-NH₂ | (SEQ ID NO: 20) |
| H-Ac₅c-Val-Aib-Glu-Ile-Gln-Leu-Met-His-Gln-Arg-Ala-Lys( )Trp-Leu-Asn-Asp( )-NH₂ | (SEQ ID NO: 25) |
| H-Ac₅c-Val-Aib-Glu-Ile-Gln-Leu-Met-His-Gln-Har-Ala-Lys( )Phe-Leu-Asn-Asp( )-NH₂ | (SEQ ID NO: 26) |
| H-Ac₅c-Val-Aib-Glu-Ile-Gln-Phe-Leu-His-Gln-Har-Ala-Lys( )Trp-Leu-Asn-Asp( )-NH₂ | (SEQ ID NO: 27) |
| H-Ac₅c-Val-Aib-Glu-Ile-Gln-Leu-Leu-His-Gln-Har-Arg-Lys( )Trp-Leu-Asn-Asp( )-NH₂ | (SEQ ID NO: 28) |
| H-Ac₅c-Val-Aib-Glu-Ile-Gln-Leu-Met-His-Gln-Har-Ala-Lys( )Trp-Leu-Asp( )-NH₂ | (SEQ ID NO: 30) |
| (H-Ac₅c-Val-Aib-Glu-Ile-Gln-Leu-Met-His-Gln-Har-Ala-Lys( )-Trp-Leu-Asp( )-NH₂)₂ | (SEQ ID NO: 33) |
| (H-Ac₅c-Val-Aib-Glu-Ile-Gln-Leu-Met-His-Gln-Har-Ala-Lys( )-Trp-Leu-Asn-Asp( )-NH₂)₂ | (SEQ ID NO: 39) |

and cyclised between amino acids in position 13 and 17, N-terminally acetylated form thereof, a C-terminal acid form thereof, a homodimer or heterodimer or a pharmaceutically acceptable salt and derivative thereof.

14. A PTH analogue peptide according to any one of claims 1 to 13 for use in therapy.

15. Use of a PTH analogue peptide according to any one of claims 1 to 13 in the preparation of a medicament for the increase of bone mass.

16. Use of PTH analogue peptide according to claim 15 in the preparation of a medicament for the treatment of osteoporosis, such as primary osteoporosis, endocrine osteoporosis (hyperthyroidism, hyperparathyroidism, Cushing's syndrome, acromegaly, type 1 diabetes mellitus, adrenal insufficiency), hereditary and congenital forms of osteoporosis (osteogenesis imperfecta, homocystinuria, Menkes' syndrome, and Riley-Day syndrome), nutritional and gastrointestinal disorders, haematological disorders/malignancy (multiple myeloma, lymphoma and leukaemia, hemophilia, thalassemia), osteoporosis due to immobilization, chronic obstructive pulmonary disease or rheumatologic disorders (rheumatoid arthritis, ankylosing spondylitis).

17. Use of PTH analogue peptide according to any of the claims 1 to 13 in the preparation of a medicament for the treatment of primary osteoporosis or endocrine osteoporosis.

18. A pharmaceutical composition comprising a PTH analogue peptide according to any one of claims 1 to 13, in combination with a pharmaceutically acceptable carrier.
